(19) United States
(12) Patent Application Publication
(10) Pub. No.: US 2025/0182845 A1
(43) Pub. Date: Jun. 5, 2025

(54) METHOD AND SYSTEM FOR PREDICTING A PROPERTY OF A HOST ORGANISM USING MICROBIOME COMPOSITION DATA

(71) Applicant: Bar-Ilan University, Ramat Gan (IL)

(72) Inventor: Yoram LOUZOUN, Elkana (IL)

(21) Appl. No.: 18/968,510

(22) Filed: Dec. 4, 2024

Related U.S. Application Data
(60) Provisional application No. 63/605,603, filed on Dec. 4, 2023.

(51) Int. Cl. G16B 20/00; G16B 10/00; G16B 40/00

(52) U.S. Cl. CPC G16B 20/00; G16B 10/00; G16B 40/00

(57) ABSTRACT

The present invention relates to the field of computational biology, specifically to methods and systems for predicting host organism properties based on microbiome composition data and metabolome interactions using machine learning techniques. The claimed invention represents a system and method for predicting one or more properties of a host organism using microbiome composition data that provide an improvement of the technological field of computational biology by increasing the accuracy and reliability of such predictions, including the prediction of metabolome concentrations based on the host's microbiome data.

### CROSS-REFERENCE TO RELATED APPLICATIONS

This application claims the benefit of priority of U.S. Provisional Patent Application No. 63/605,603, filed Dec. 4, 2023, the contents of which are all incorporated herein by reference in their entirety.

### FIELD OF THE INVENTION

The present invention relates to the field of computational biology, specifically to methods and systems for predicting host organism properties based on microbiome composition data and metabolome interactions using machine learning techniques.

### BACKGROUND OF THE INVENTION

In the field of computational biology, it is well-established that the composition and interactions of microbial communities can significantly influence the host organism properties (e.g., biological or clinical properties). These interactions are often mediated through the production and consumption of metabolites, which can affect various biological processes within the host.

Specifically, the human gut microbial composition is associated with multiple aspects of human health. The microbiome is associated with human health, either directly through the effect of microbes on disease, or indirectly through interaction with different systems of the human host. However, the most extensive interaction with the host is through metabolite consumption and production with short-chain fatty acids (SCFAs) such as butyrate, acetate and propionate, the end product of gut microbiome fermentation, being some of the most studied metabolites. SCFAs have been shown to have a role in regulating the immune response and gut barrier function, gut cell proliferation and differentiation, regulation of gut endocrine functions and even in gut brain axis communication. The relation between metabolites and microbes is bi-directional, with each affecting the frequency/concentration of the other. However, typically, the prediction was from the microbiome to the metabolites and not vice-versa. Indeed, metabolites have been shown to be affected by heritable, gut microbiome, by lifestyle choices such as smoking, diet etc.

Both microbiome and metabolome have been associated with the host condition through either correlations or predictions, often in conjunction with additional meta-data, such as age, gender or dict. Such predictions typically require machine-learning (ML)-based models, e.g., including Deep Neural Networks (DNNs) and Convolutional Neural Networks (CNNs). However, inferring the human condition based on either microbiome or metabolome separately suffers from several limitations. The limitations of microbiome-based ML include among others: little knowledge about the interaction between different members of the microbial community or with the host, and the absence of mechanistic understanding of the relation between the microbiome and health or disease. Microbiome-based ML is also often plagued by a low prediction accuracy if compared to results obtained based on other sources of information, such as metabolites.

While metabolome studies have become increasingly used in characterizing emerging properties of the metabolome and in relating metabolomic change to host pathological states, metabolome based ML also has its limitations, such as: high cost; extremely high dimension of input (i.e., number of different metabolites vs. the number of samples), especially in untargeted studies; a large number of unknown metabolites that have a molecular composition, but no known function; and large variability of nomenclature and experimental protocols among different studies.

### SUMMARY OF THE INVENTION

Accordingly, there is a need for a system and method for predicting one or more properties of a host organism using microbiome composition data that would provide an improvement of the technological field of computational biology by increasing the accuracy and reliability of such predictions, including the prediction of metabolome concentrations based on the host's microbiome data. Additionally, there is a need for a solution that accurately leverages the complex, non-linear, and bi-directional interactions between the microbiome and metabolome of the host. This solution should consider the dynamic relationships between different microbial species and their collective impact on the host's metabolic profile.

In the general aspect, the invention may be directed to a method for predicting at least one property of a host organism, by at least one processor. The method may include: analyzing a biological sample of the host organism to obtain a microbiome composition data element comprising information about frequency values of various microbial taxa characterizing the biological sample; preprocessing the microbiome composition data element by grouping and normalizing said frequency values, based on a microbial taxonomic classification; obtaining an intermediate latent representation of the preprocessed microbiome composition data element by embedding the preprocessed microbiome composition data element into a latent feature space using a fully connected neural network (FCNN), said latent feature space being representative of a microbiome-metabolome interaction; and predicting said at least one property of the host organism, based on the obtained intermediate latent representation.

In another general aspect, the invention may be directed to a system for predicting at least one property of a host organism. The system may include at least one non-transitory memory device, wherein modules of instruction code are stored, and at least one processor associated with said at least one memory device, and configured to execute the modules of instruction code. Upon execution of said modules of instruction code, the at least one processor may be configured to: analyze a biological sample of the host organism to obtain a microbiome composition data element comprising information about frequency values of various microbial taxa characterizing the biological sample; preprocess the microbiome composition data element by grouping and normalizing said frequency values, based on a microbial taxonomic classification; obtain an intermediate latent representation of the preprocessed microbiome composition data element by embedding the preprocessed microbiome composition data element into a latent feature space using a fully connected neural network (FCNN), said latent feature space being representative of a microbiome-metabolome interaction; and predict said at least one property of the host organism, based on the obtained intermediate latent representation.

In some embodiments, the preprocessing may include: selecting a taxonomy level of a microbial taxonomic classification; merging frequency values of microbial taxa belonging to a same taxonomic group of the selected taxonomy level; based on the merged frequency values, applying Principal Component Analysis (PCA) on each taxonomic group of the selected taxonomy level, to determine those of the microbial taxa that explain at least half of a variance in the merged frequency values; and forming the preprocessed microbiome composition data element, based on the determined microbial taxa.

Accordingly, in some embodiments, the at least one processor may be configured to preprocess the microbiome composition data element by: selecting a taxonomy level of a microbial taxonomic classification; merging frequency values of microbial taxa belonging to a same taxonomic group of the selected taxonomy level; based on the merged frequency values, applying Principal Component Analysis (PCA) on each taxonomic group of the selected taxonomy level, to determine those of the microbial taxa that explain at least half of a variance in the merged frequency values; and forming the preprocessed microbiome composition data element, based on the determined microbial taxa.

In some embodiments, the stage of forming the preprocessed microbiome composition data element may include applying a logarithmic normalization to frequency values of the determined microbial taxa, so as to reduce the impact of highest frequency values and prevent zero frequency values.

Accordingly, in some embodiments, the at least one processor may be configured to form the preprocessed microbiome composition data element by applying a logarithmic normalization to frequency values of the determined microbial taxa, so as to reduce the impact of highest frequency values and prevent zero frequency values.

In some embodiments, the latent feature space may include latent microbiome features mapped to the concentration of one or more metabolites by a predefined function.

In some embodiments, said at least one property may include a metabolite concentration. In some embodiments, predicting of the at least one property may include determining a metabolite concentration of the host organism by applying an approximated microbiome-metabolite relationship matrix to the intermediate latent representation, said approximated microbiome-metabolite relationship matrix comprising parameters of said predefined function.

Accordingly, in some embodiments, said at least one processor may be configured to predict said at least one property by determining the metabolite concentration of the host organism by applying an approximated microbiome-metabolite relationship matrix to the intermediate latent representation, said approximated microbiome-metabolite relationship matrix comprising parameters of said predefined function.

In some embodiments, said predefined function may be a linear function from the features of the latent feature space to logarithmically normalized values of said metabolite concentration.

In some embodiments, said stage of applying of the approximated microbiome-metabolite relationship matrix may include multiplying the intermediate latent representation by the approximated microbiome-metabolite relationship matrix, to obtain said logarithmically normalized values of said metabolite concentration.

In some embodiments, said stage of predicting the at least one property may include inferring a machine-learning (ML)-based condition prediction model on the intermediate latent representation, said ML-based condition prediction model being pretrained to predict said at least one property based on the latent microbiome features.

Accordingly, in some embodiments, the at least one processor may be configured to predict said at least one property by inferring a machine-learning (ML)-based condition prediction model on the intermediate latent representation, said ML-based condition prediction model being pretrained to predict said at least one property based on the latent microbiome features.

In some embodiments, said property may be determined by a binary value and the ML-based condition prediction model may be a logistic regression model; or said property may be determined by a continuous value and the ML-based condition prediction model may be a Ridge regression model.

In some embodiments, the approximated microbiome-metabolite relationship matrix may be obtained by the following procedure: (a) based on a training set of paired training samples comprising training microbiome composition data elements and training metabolite concentration data elements, training the FCNN to determine the latent feature space, while concurrently determining an initial microbiome-metabolite relationship matrix comprising parameters of an initial function mapping (i) inverse latent representation matrices obtained by applying a pseudo-inverse algorithm on intermediate latent representations of training microbiome composition data elements, said intermediate latent representations of training microbiome composition data elements being calculated by embedding said training microbiome composition data elements into the determined latent feature space; to (ii) training metabolite concentration data elements of respective paired training samples; and (b) applying a low-rank approximation of the initial microbiome-metabolite relationship matrix using Singular Value Decomposition (SVD) algorithm, to obtain the approximated microbiome-metabolite relationship matrix.

In some embodiments, the method may further include the following: based on a training set of paired training samples comprising training microbiome composition data elements and training metabolite concentration data elements, training the FCNN to determine the latent feature space assuming a correlation with an initial function mapping (i) inverse latent representation matrices obtained by applying a pseudo-inverse algorithm on intermediate latent representations of training microbiome composition data elements, said intermediate latent representations of training microbiome composition data elements being calculated by embedding said training microbiome composition data elements into the determined latent feature space; to (ii) training metabolite concentration data elements of respective paired training samples.

In some embodiments, the method may further include concurrently determining an initial microbiome-metabolite relationship matrix comprising parameters of said initial function.

In some embodiments, said microbiome composition data element is represented by at least one of (i) 16S rRNA gene sequencing; and (ii) Whole Genome Shotgun Sequencing (WGS).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a block diagram illustrating training stage aspects, according to the concept of the present invention in some embodiments thereof;

FIG. 1B is a block diagram illustrating inference stage aspects, according to the concept of the present invention in some embodiments thereof;

FIG. 2 is a block diagram depicting a computing device which may be included in a system for predicting at least one property of the host organism, according to some embodiments;

FIG. 3A is a block diagram, depicting the system for predicting at least one property of the host organism, according to some embodiments;

FIG. 3B is a block diagram, depicting, in detail, a preprocessing module of the system for predicting at least one property of the host organism, according to some embodiments;

FIG. 3C is a block diagram, depicting, in detail, a training module of the system for predicting at least one property of the host organism, according to some embodiments;

FIG. 4 is a flow diagram, depicting a method for predicting at least one property of the host organism, according to some embodiments;

FIG. 5 is a diagram, schematically illustrating different approaches to microbiome-metabolites relations, according to some embodiments;

FIGS. 6A-6B are histograms of the coefficients of the NMF model which relates metabolite concentrations and the microbiome frequencies (real in dark purple) and of a random model with the microbes shuffled before the prediction (light purple).

FIGS. 6C-6D are swarm plots of all the expectations of the relative contribution of the coefficients of each metabolite for all the 16S rRNA gene-based (FIG. 6C) and the WGS datasets (FIG. 6D).

FIGS. 6E-6F are bar plots of the frequency of the microbes associated with the 10 highest coefficients in the NMF models of C5H11NO2S (FIG. 6E) and C4H7NO4 (FIG. 6F);

FIG. 6G is a scatter plot of the coefficients in the log NMF model of the taxa with the highest coefficients vs. the logged frequency of the same taxa, with no clear correlation between them;

FIGS. 7A-7D are charts illustrating comparison between LOCATE and the state-of-the-art metabolites prediction models over the different 16S datasets “He” (7A), “Poyet” (7B), “Jacob” (7C), and “Direct Plus” (7D).

FIGS. 7E-7F are bar charts illustrating average SCCs over all tested metabolites and all tested datasets per model, the 16S averages (FIG. 7E) and the WGS averages (FIG. 7F).

FIG. 8A is a heatmap of significant SCCs between microbes and SCFA over different WGS datasets (“ERAWIJANTARI”, “FRANZOSA”, “MARS”, “WANG”, “YACHIDA”).

FIG. 8B is a heatmap of significant SCCs between all common microbes and metabolites over different gastric problems WGS datasets (“ERAWIJANTARI”, “FRANZOSA”, “MARS”, “YACHIDA”).

FIG. 8C is a heatmap of SCC between microbes and metabolites over different datasets (“He”, “Kim”, and “Jacob”) vs. the relations that are reported in the literature.

FIG. 8D are bar charts illustrating the core microbiome, covering about 20 orders which are common to most of the datasets.

FIG. 8E is a swarm plot of LOCATE's predicted metabolites SCCs in the cross-times test over the “Direct Plus” cohort;

FIGS. 8F-8H are swarm plots of all tested cross-datasets predictions between couples of datasets on the shared metabolites and microbes, “He-Direct Plus” (FIG. 8F), “He-Kim” (FIG. 8G), “He-Jacob” (FIG. 8H);

FIG. 9A is a bar chart illustrating average SCC between the CCA outputs of the microbiome and metadata (pink), the metabolites and metadata (yellow), and LOCATE's representation and the metadata (blue);

FIGS. 9B-9D are bar chart of weights of the CCA between LOCATE's representations and the metadata on its two first components on “He” (FIG. 9B), “Jacob” (FIG. 9C) and “Poyet” (FIG. 9D).

FIGS. 9E-9F are bar plots of average AUC (FIG. 9E) and the average SCC (FIG. 9F) of the predicted outcomes over different datasets and different tasks;

FIGS. 9G-9I are charts demonstrating effect of a decreasing number of metabolites for LOCATE's representation on the condition predictions in “He” (FIG. 9G), “Jacob” (FIG. 9H) and “Poyet” (FIG. 9I).

FIGS. 9J-9K are bar plots of average AUC (FIG. 9J) and the average SCC (FIG. 9K) of the predicted outcomes over different datasets and different tasks;

FIGS. 10A-10I is a set of charts showing that internal representation improves outcome prediction compared with the microbiome and when possible also metabolites and is associated with datasets features, following the structure of FIGS. 9A-9K, but for WGS datasets.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, “choosing”, “selecting”, “omitting”, “training”, “applying”, “forming” or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

It shall be understood, that, in the present description, the system for predicting at least one host property according to the present invention is also referred herein as a “Latent variables Of microbiome And meTabolites rElaTions” system or “LOCATE”.

It shall be understood that, in the context of present invention, terms like ‘microbiome’, ‘microbiome composition’, ‘microbiome data’, ‘microbial species’, ‘microbe’, ‘microbiome composition data element’ and similar, in the context of the present invention, shall be considered equivalent. Furthermore, in some contexts, amplicon sequence variant (ASV) may refer to ‘microbiome composition data element’ and therefore may be considered equivalent.

It shall be understood that, in the context of present invention, terms like ‘metabolome’, ‘metabolite concentrations’, ‘metabolome concentrations’, ‘metabolome data’, ‘metabolic profile’, ‘metabolome concentration data element’ and similar, in the context of the present invention, shall be considered equivalent.

It shall be understood that, in the context of present invention, terms like ‘host property’, ‘host condition’, ‘host characteristics’ and similar, in the context of the present invention, shall be considered equivalent.

It shall be understood that, in the context of present invention, terms like ‘latent representation’, ‘intermediate latent representation’ or simply ‘representation’ and similar, in the context of the present invention, shall be considered equivalent.

It shall be understood that, in the context of present invention, terms like ‘logistic regression model’ may refer to ‘ML-based condition prediction model’ and, in the context of the present invention, these terms may be considered equivalent.

The list of explained acronyms used in the present disclosure is provided below:
LOCATE—Latent variables Of microbiome And meTabolites rElations;
ML—Machine Learning
SCFA—Short Chain Fatty Acids;
TID—Type 1 Diabetes;
IBD—Inflammatory Bowel Disease;
T2D—Type 2 Diabetes;
DNN—Deep Neural Networks;
CNN—Convolutional Neural Networks;
PRMT—Predicted Relative Metabolomic Turnover;
MIMOSA—Model-based Integration of Metabolite Observations and Species Abundance;
MLPNN—Multiple-Layer Perceptron Neural Network;
WGS—Whole Genome Shotgun Sequencing;
HDG—Healthy Dietary Guidelines;
MRS—Magnetic Resonance Spectroscopy;
DSC—Deep Subcutaneous;
SSC—Superficial Subcutaneous;
VAT—Visceral Adipose Tissue;
CD—Crohn's Disease;
UC—Ulcerative Colitis;
ESRD—End-Stage Renal Disease;
NMF—Non Negative Matrix Factorization;
NNI—Neural Network Intelligence;
MSE—Mean Square Error;
SCC—Spearman Correlation Coefficient;
AUC—Area Under the ROC Curve;
CCA—Canonical-Correlation Analysis;
SVD—Singular Value Decomposition;
CRC—Colorectal Cancer.

Unless explicitly stated, the method embodiments described herein are not constrained to a particular order or sequence. Additionally, some of the described method embodiments or elements thereof can occur or be performed simultaneously, at the same point in time, concurrently, or iteratively and repeatedly.

In embodiments of the present invention, some steps of the claimed method may be performed using machine-learning (ML)-based models or may include actions performed on ML-based models, e.g., transferring ML-based models over a computer network. ML-based models may be configured or “trained” for a specific task, e.g., classification or regression.

In some embodiments, ML-based models may be artificial neural networks (ANN).

A neural network (NN) or an artificial neural network (ANN), e.g., a neural network implementing a machine learning (ML) or artificial intelligence (AI) function, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g., CPUs or graphics processing units (GPUs), or a dedicated hardware device may perform the relevant calculations.

It should be obvious for the one ordinarily skilled in the art that various ML-based models can be implemented without departing from the essence of the present invention. It should also be understood, that in some embodiments ML-based model may be a single ML-based model or a set (ensemble) of ML-based models realizing as a whole the same function as a single one. Hence, in view of the scope of the present invention, the abovementioned variants should be considered equivalent.

To understand the improvements that the present invention provides, one can contrast simple models of microbiome-metabolome relations known in the art and listed below (schematically demonstrated in FIG. 5).
A) Linear approach. Linear model is focused on metabolite production. Each microbe produces metabolites, and the metabolite concentration is a positive linear combination of these microbe productions. The metabolite concentration can thus be described by a non-negative factorization of the microbe frequencies, which would basically capture the contribution of each microbe to each metabolite studied. Some studies propose qualitative relations, where each microbe is associated with high or low values of a metabolite. Those are mostly based on biological relations of production and consumption. Other studies propose quantitative relations. Some of those are reference-based, such as Predicted Relative Metabolomic Turnover (PRMT), MIMOSA (Model-based Integration of Metabolite Observations and Species Abundances), and Mangosteen, while others are model-based, such as: MelonnPan (FIG. 5, section A).
B) Dominant microbes approach. An alternative hypothesis to the linear model would be that given the dominance of a small number of microbe species composing the vast majority of microbes in the gut, the concentration of each metabolite is determined by the most frequent microbe. This conceptualization translates into models that relate one main microbe (or set of genetically similar microbes) to each metabolite (FIG. 5, section B). While this is not implemented in any quantitative model. This is the assumption underlying most qualitative arguments suggesting that changing a dominant microbe would change the metabolite concentration.
C) Multiview approach. Contrary to the 2 former approaches, which assume the microbiome and metabolites interactions are direct and the environment affects the situation via the microbiome only, this model assumes the microbiome and metabolites are both affected by the environment. Therefore, the conditions of the samples (which determine the environment for the microbiome and metabolome) can be estimated from the microbiome and metabolites by using multi-omics approaches, such as Multiview and IntegratedLearner. Despite its multi-faceted approach, this model falls short of creating a learnable connection between the microbiome and metabolites (FIG. 5, section C).
D) Latent variables model. It is suggested herein to follow a model where the observed frequencies of microbes and concentrations of metabolites represent the steady state of complex bi-directional interactions. In such a case, the effect of a microbe on metabolites is not linearly or positively correlated with its frequency. Moreover, the equilibrium is affected by the environment (e.g. heritability, lifestyle choices, and diet) and differs between hosts. While this complicates modeling the relationship between metabolites and microbiome, it produces a latent representation of the relation. This representation can then be directly associated with the environment (FIG. 5, section D). It is proposed herein that this approach is indeed better than the ones above for predicting the relation between microbes, metabolites, and the host condition.

There are also more heuristic ML-based models that do not fit clearly into these categories, such as MiMeNet, and encoder-decoder-based models, such as SparseNED, mNODE, and the model proposed by Khajeh et al. that showed that autoencoders of microbiome and metabolome can be used for IBD prediction.

The proposed method addresses the limitations of the prior-art methods by treating the microbiome-metabolome relationship as the equilibrium of a complex interaction. This method relates the host condition to a latent representation of the interaction between the log concentration of the metabolome and the log frequencies of the microbiome. The method involves analyzing a biological sample of the host organism to obtain microbiome composition data, preprocessing this data by grouping and normalizing frequency values based on microbial taxonomic classification, and embedding the preprocessed data into a latent feature space using a fully connected neural network. This latent representation is then used to predict the host property, providing a more accurate and reliable prediction compared to existing methods. The proposed method improves the prediction of metabolite concentrations from microbiome composition and enhances the prediction of host conditions by leveraging the latent representation of microbiome-metabolome interactions.

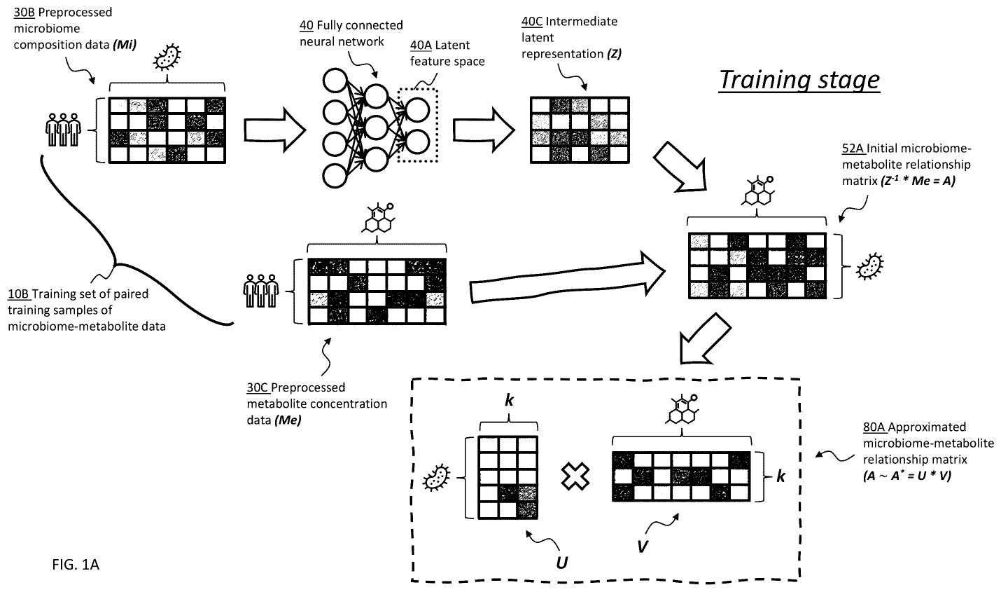

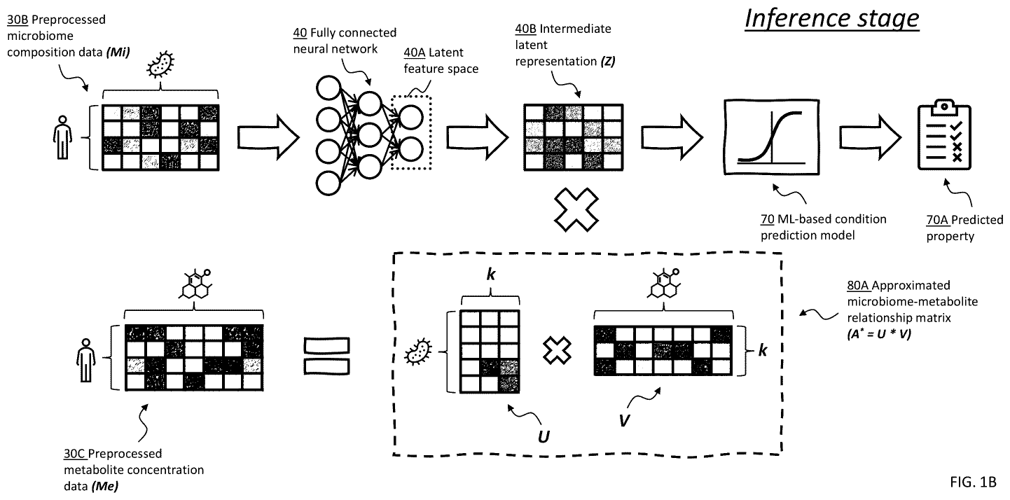

The concept of the present invention is elaborated in greater detail with reference to FIGS. 1A and 1B.

FIG. 1A shows aspects of the training stage, that, in some embodiments, may be applied to determine elements and parameters of the system for predicting at least one property of the host organism, as suggested herein.

For the training, a training set 10B of paired training samples of preprocessed microbiome-metabolite data (denoted Mi and Me correspondingly) is used as an input. During training, the preprocessed microbiome data is projected to an intermediate latent representation 40C (also denoted ‘Z’) having a lower dimension than the microbiome using a fully connected neural network (FCNN) 40. Then ‘Z’ is used to predict the metabolites of the training set (of a respective paired samples). System finds an initial microbiome-metabolite relationship matrix 52A (also denoted herein as ‘A’), using an initial function (which may be a predefined function, e.g., linear function). For example, in some embodiments, such the initial function may be Z⁻¹*Me=A. Training process is done iteratively, using different samples of dataset 10B. As a result of the training, network 40 forms latent feature space 40A that includes (i) latent microbiome features mapped to the concentration of one or more metabolites by a predefined function, e.g., have a linear correlation therewith; and (ii) relationship matrix ‘A’ that stores the parameters of the function. As training relates to paired samples of microbiome-metabolome data, the obtained latent microbiome features inherently reflect complex dependencies between the two. This entire training process is performed at once, concurrently determining matrix ‘A’ and feature space 40A.

After the training is completed, matrix ‘A’ may then be passed through a low-rank approximation algorithm (e.g., using SVD) to prevent an overfit, thereby obtaining an approximated microbiome-metabolite relationship matrix 80A (also denoted herein as ‘A*’), e.g., such that A˜A*=U*V.

FIG. 1B shows aspects of the inference stage, that, in some embodiments, may be applied to determine a metabolite concentration of the host organism and/or other properties of the host organism, which may be or may include, e.g., biological properties (e.g., including age, gender, or various physiological properties), clinical properties (e.g., deceases, allergies, pathologies, etc.) and so on, including any other properties that may be connected to or affected by microbiome characteristics of the host organism, metabolome characteristics of the host organism, or both.

According to the concept of the present invention, at the inference stage, preprocessed microbiome composition data element 30B of the target host organism may be passed through the pretrained network 40, to obtain intermediate latent representation 40B of the preprocessed microbiome composition data element 30B by embedding the preprocessed microbiome composition data element 30B into latent feature space 40A that was formed during the training stage, as described above. Then, approximated microbiome-metabolite relationship matrix 80A that was obtained during the training stage may be applied to intermediate latent representation 40B (e.g., in case of the linear predefined function, may be multiplied thereby), to obtain (predict) preprocessed metabolite concentration data 30C of the host organism. In some additional or alternative embodiments, latent representation 40B may be used as an input to ML-based condition prediction model 70, alone or in combination with other inputs (e.g., diet, habits etc.) to predict the desired property 70A of the host organism. Accordingly, ML-based condition prediction model 70 may be pretrained to predict said at least one property 70A based on the latent microbiome features.

Therefore, the present invention offers a more accurate and reliable prediction of host properties based on microbiome data than the known solutions. This is achieved through the use of latent representations and advanced machine learning techniques, which together address the limitations of traditional models and enhance the understanding of microbiome-metabolome interactions.

The efficiency of the suggested techniques have been tested and approved, as further explained in greater detail.

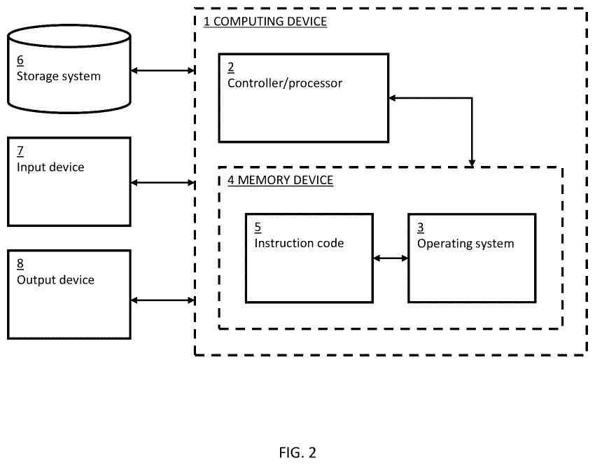

Reference is now made to FIG. 2, which is a block diagram depicting a computing device, which may be included within an embodiment of the system for secure distribution of ML-based models, according to some embodiments.

Computing device 1 may include a processor or controller 2 that may be, for example, a central processing unit (CPU) processor, a chip or any suitable computing or computational device, an operating system 3, a memory device 4, instruction code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to instruction code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It is noted that an operating system 3 may be an optional component, e.g., in some embodiments, a system may include a computing device that does not require or include an operating system 3.

Memory device 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory device 4 may be or may include a plurality of possibly different memory units. Memory device 4 may be a computer or processor non-transitory readable medium, or a computer non-transitory storage medium, e.g., a RAM. In one embodiment, a non-transitory storage medium such as memory device 4, a hard disk drive, another storage device, etc. may store instructions or code which when executed by a processor may cause the processor to carry out methods as described herein.

Instruction code 5 may be any executable code, e.g., an application, a program, a process, task, or script. Instruction code 5 may be executed by processor or controller 2 possibly under control of operating system 3. For example, instruction code 5 may be a standalone application or an API module that may be configured to analyze a biological sample of the host organism to obtain a microbiome composition data element comprising information about frequency values of various microbial taxa characterizing the biological sample; preprocess the microbiome composition data element by grouping and normalizing said frequency values, based on a microbial taxonomic classification; obtain an intermediate latent representation of the preprocessed microbiome composition data element by embedding the preprocessed microbiome composition data element into a latent feature space using a fully connected neural network (FCNN), said latent feature space being representative of a microbiome-metabolome interaction; and predict said at least one property of the host organism, based on the obtained intermediate latent representation, as further described herein. Although, for the sake of clarity, a single item of instruction code 5 is shown in FIG. 2, a system according to some embodiments of the invention may include a plurality of executable code segments or modules similar to instruction code 5 that may be loaded into memory device 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Various types of input and output data may be stored in storage system 6 and may be loaded from storage system 6 into memory device 4 where it may be processed by processor or controller 2. In some embodiments, some of the components shown in FIG. 2 may be omitted. For example, memory device 4 may be a non-volatile memory having the storage capacity of storage system 6. Accordingly, although shown as a separate component, storage system 6 may be embedded or included in memory device 4.

Input devices 7 may be or may include any suitable input devices, components, or systems, e.g., sensors configured to measure parameters of the biological sample, e.g., gut microbiome sample. Input devices 7 may also include, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

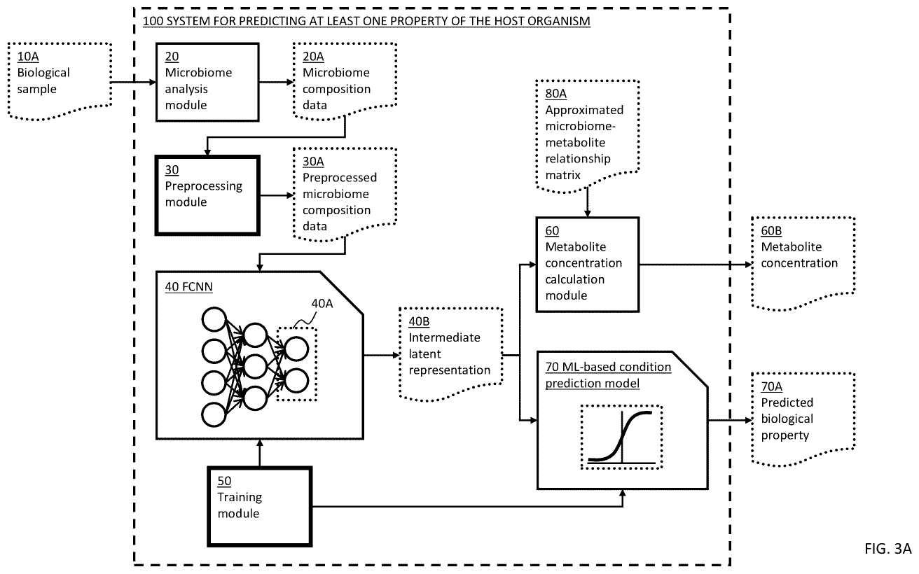

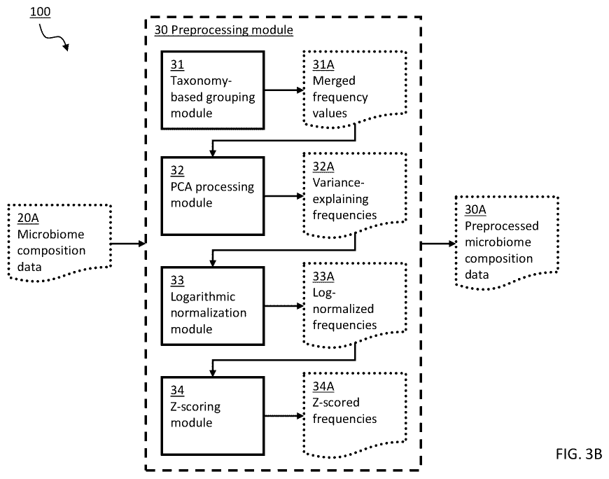

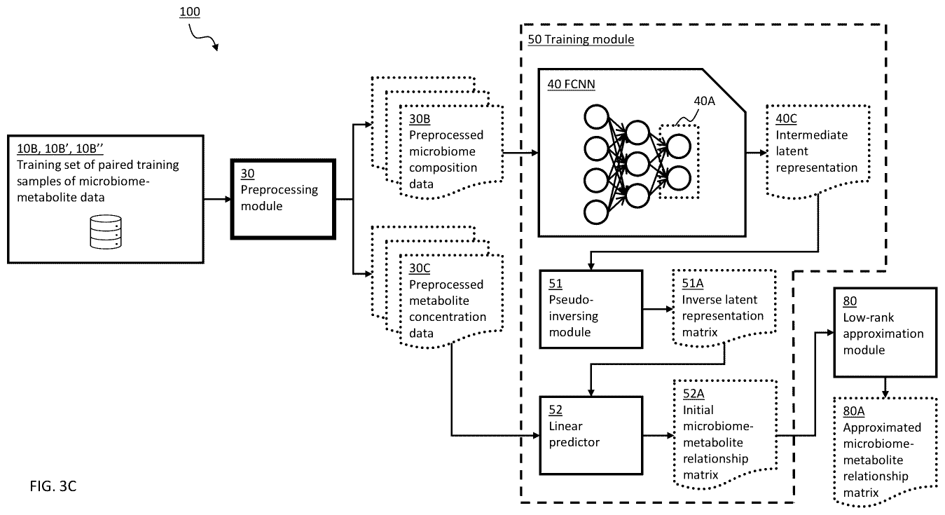

Reference is now made to FIGS. 3A-3C complementary depicting various aspects of system 100 for predicting at least one property of the host organism, according to some embodiments.

According to some embodiments of the invention, system 100 may be implemented as a software module, a hardware module, or any combination thereof. For example, system 100 may be or may include computing devices such as element 1 of FIG. 2. Furthermore, system 100 may be adapted to execute one or more modules of instruction code (e.g., element 5 of FIG. 2) to request, receive, analyze, calculate and produce various data.

As further described in detail herein, system 100 may be adapted to execute one or more modules of instruction code (e.g., element 5 of FIG. 1) in order to perform steps of the claimed method.

As shown in FIGS. 3A-3C, arrows may represent the flow of one or more data elements to and from system 100 and/or among modules or elements of system 100. Some arrows have been omitted in FIGS. 3A-3C for the purpose of clarity.

As shown in FIG. 3A, in some embodiments, system 100 may include microbiome analysis module 20. Microbiome analysis module 20 may be configured to receive biological sample 10A (e.g., gut microbiome sample). Module 20 may be further configured to analyze biological sample 10A of the host organism to obtain microbiome composition data element 20A comprising information about frequency values of various microbial taxa characterizing the biological sample. E.g., in some embodiment, module 20 may be configured to generate microbiome composition data element 20A represented by at least one of (i) 16S rRNA gene sequencing; and (ii) Whole Genome Shotgun Sequencing (WGS).

In some embodiments, system 100 may further include preprocessing module 30, which is further described in greater details with reference to FIG. 3B.

In some embodiments, preprocessing module 30 may be configured to receive microbiome composition data element 20A. Preprocessing module 30 may include: taxonomy-based grouping module 31, PCA processing module 32, logarithmic normalization module 33 and Z-scoring module 34.

In some embodiments, taxonomy-based grouping module 31 may be configured to receive microbiome composition data element 20A, select a desired taxonomy level of microbial taxonomic classification (the relevant configuration may be preset in system 100 or defined dynamically), and merge frequency values of microbial taxa belonging to the same taxonomic group of the selected taxonomy level, thereby outputting merged frequency values 31A.

In some embodiments, PCA processing module 32 may be configured to receive merged frequency values 31A and, based on the merged frequency values, apply Principal Component Analysis (PCA) on each taxonomic group of the selected taxonomy level, to determine those of the microbial taxa that explain at least half of a variance in the merged frequency values, thereby outputting variance-explaining frequencies 32A. It shall be understood that PCA processing is provided herein as a non-exclusive example only, and other processing methods have been tested and may be applied herein without going beyond the scope of the present invention. E.g., in some additional or alternative embodiments, said preprocessing may be performed by averaging frequency values, based on taxonomic group and the selected taxonomy level.

In some embodiments, logarithmic normalization module 33 may be configured to receive variance-explaining frequencies 32A and apply a logarithmic normalization to frequency values 32A of the determined microbial taxa, so as to reduce the impact of highest frequency values and prevent zero frequency values, thereby outputting log-normalized frequency values 33A.

In some embodiments, Z-scoring module 34 may be configured to receive log-normalized frequencies 33A and to further normalize each taxon such that its average will be 0 and its variance will be 1 (that is, perform the known z-scoring algorithm), thereby outputting Z-scored frequencies 34A, which may be used to form preprocessed microbiome composition data element 30A, as an output of preprocessing module 30.

Referring again to FIG. 3A, it is further shown that, in some embodiments, system 100 may further include the discussed above ML-based model for determining intermediate latent representation 40B—fully-connected neural network (FCNN) 40.

In some embodiments FCNN 40 may be configured to perform embedding of the preprocessed microbiome composition data element 30A into latent feature space 40A (that may be obtained during training, as discussed with reference to FIG. 1A above, and as further explained with reference to FIG. 3C). Latent feature space 40A may be representative of a microbiome-metabolome interaction. Thereby, FCNN 40 may generate intermediate latent representation 40B of the preprocessed microbiome composition data element 30A.

The specific configuration of the FCNN 40, that may be applied in some embodiments of the present invention, is described further below, when referring to the tests conducted to evaluate reliability of the techniques suggested herein.

In some embodiments system 100 may further include metabolite concentration calculation module 60. Metabolite concentration calculation module 60 may be configured to receive intermediate latent representation 40B and approximated microbiome-metabolite relationship matrix 80A. Approximated microbiome-metabolite relationship matrix 80A may be obtained during training stage (as explained with reference to FIG. 1A above, and as further explained with reference to FIG. 3C), and may be stored in the memory device of system 100 (such as memory device 4 of FIG. 2).

Metabolite concentration calculation module 60 may be further configured to determine metabolite concentration 60B of the host organism by applying approximated microbiome-metabolite relationship matrix 80A to intermediate latent representation 40B.

In some embodiments, latent feature space 40A may include latent microbiome features mapped to the concentration of one or more metabolites by a predefined function, wherein said latent features may be determined during the training stage, as explained above. In some embodiments, said approximated microbiome-metabolite relationship matrix 80A may store parameters of this predefined function, also determined during the training stage.

E.g., in some embodiments, the predefined function may be a linear function from the features of latent feature space 40A to logarithmically normalized values of said metabolite concentration 60B. E.g., predefined function may be Me=A^{*}*Z, as explained with reference to FIG. 1B.

Accordingly, in some embodiments, module 60 may be configured to multiply intermediate latent representation 40B by approximated microbiome-metabolite relationship matrix 80A, to obtain said logarithmically normalized values of said metabolite concentration 60B.

In some further embodiments, system 100 may include ML-based condition prediction model 70. In some embodiments, model 70 may be pretrained to predict said at least one property of the host based on the latent microbiome features of the feature space 40A. Accordingly, model 70 may be configured to receive intermediate latent representation 40B. System 100 may be configured to infer model 70 on representation 40B, thereby predicting the target property 70A. E.g., in some non-exclusive embodiments, said property 70A may be determined by a binary value and ML-based condition prediction model 70 may be a logistic regression model; or said property 70A may be determined by a continuous value and ML-based condition prediction model 70 may be a Ridge regression model. It shall be understood that other architectures of model 70 may be applied herein without going beyond the scope of the present invention.

In some embodiments, system 100 may further include training module 50, connected to both FCNN 40 and model 70, and configured to train them.

Aspects of training are further explained with reference to FIG. 3C.

As can be seen in FIG. 3C, for the matter of training, system 100 may be configured to receive training dataset 10B, of paired training samples comprising training microbiome composition data elements 10B′ (such as data element 10A, explained with reference to FIG. 3A) and training metabolite concentration data elements 10B″ (e.g., comprising values of measured concentrations of various metabolites).

For microbiome composition data elements 10B′, preprocessing module 30 may be configured to perform same preprocessing procedure, as explained with reference to FIG. 3B, thereby obtaining a set of preprocessed microbiome composition data elements 30B.

For metabolome composition data elements 10B″, preprocessing module 30 may be configured to normalize concentration values to relative frequencies, such that the metabolites of each sample would sum to 1. Then, module 30 may further apply z-scoring to the log-normalized metabolite concentration values, such that the average value of each metabolite would be 0 and its variance would be 1. Accordingly, preprocessing module 30 may be configured to output a set of preprocessed metabolite concentration data elements 30C, paired to elements 30B respectively.

In some embodiments, training module 50 may be configured to receive paired sets of data elements 30B and 30C. Training module 50 may be configured to infer FCNN 40 on each of data elements 30B, thereby obtaining intermediate latent representations 40C thereof. Training module 50 may further include pseudo-inversing module 51, configured to apply a pseudo-inversing algorithm (e.g., known-in-the-art one), thereby converting intermediate latent representations 40C to inverse latent representation matrices 51A (also denoted herein as Z⁻¹).

In some embodiments, training module 50 may further include linear predictor 52, configured to apply the initial function (e.g., linear function, such as Z⁻¹*Me=A, as explained with reference to FIG. 1B), using preprocessed metabolite concentration data elements 30C and inverse latent representation matrices 51A, corresponding to the paired training samples, thereby calculating initial microbiome-metabolite relationship matrices 52A.

Accordingly, in some embodiments, training module 50 may be further configured to, train FCNN 40 to determine latent feature space 40A assuming a correlation with an initial function (e.g., linear function, such as Z⁻¹*Me=A, as explained with reference to FIG. 1B) mapping (i) inverse latent representation matrices 51A obtained by applying the pseudo-inverse algorithm on intermediate latent representations 40C of training microbiome composition data elements 10B′ (e.g., obtained based on data elements 30B), said intermediate latent representations 40C of training microbiome composition data elements 10B′ being calculated by embedding said training microbiome composition data elements 10B′ (or, in this example, preprocessed microbiome composition data elements 30B) into the determined latent feature space 40A; to (ii) training metabolite concentration data elements 10B″ (or, in this example, preprocessed metabolite concentration data elements 30C) of respective paired training samples. Accordingly, during training of FCNN 40, training module 50 may be configured to concurrently determine initial microbiome-metabolite relationship matrix 52A comprising parameters of said initial function, using linear predictor 52.

In some embodiments, system 100 may further include low-rank approximation module 80. Low-rank approximation module 80 may be configured to receive the generated initial microbiome-metabolite relationship matrix 52A and perform a low-rank approximation of initial microbiome-metabolite relationship matrix 52A using Singular Value Decomposition (SVD) algorithm (according to known-in-the-art techniques), to obtain approximated microbiome-metabolite relationship matrix 80A.

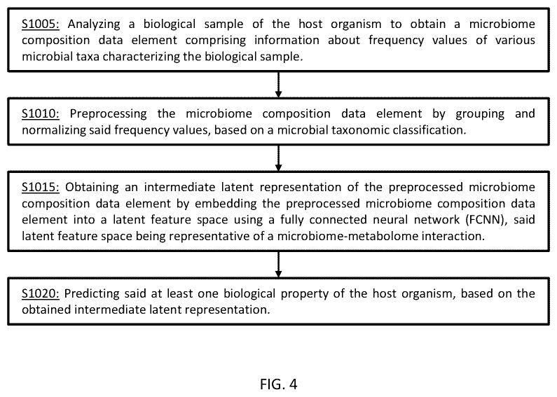

Referring now to FIG. 4, a flow diagram is presented, depicting a method for predicting at least one property of the host organism, by at least one processor (e.g., processor 2 of FIG. 2), according to some embodiments.

As shown in step S1005, the at least one processor (e.g., such as processor 2 of FIG. 2) may analyze a biological sample (e.g., biological sample 10A, as shown in FIG. 3A) of the host organism to obtain a microbiome composition data element (e.g., data element 20A, as shown in FIG. 3A) comprising information about frequency values of various microbial taxa characterizing the biological sample (e.g., biological sample 10A, as shown in FIG. 3A). Step S1005 may be carried out by microbiome analysis module 20 (as described with reference to FIG. 3A).

As shown in step S1010, the at least one processor (e.g., such as processor 2 of FIG. 2) may preprocess the microbiome composition data element (e.g., data element 20A, as shown in FIG. 3A) by grouping and normalizing said frequency values, based on a microbial taxonomic classification. Step S1010 may be carried out by preprocessing module 30 (as described with reference to FIGS. 3A-3B).

As shown in step S1015, the at least one processor (e.g., such as processor 2 of FIG. 2) may obtain an intermediate latent representation (e.g., representation 40B, as shown in FIG. 3A) of the preprocessed microbiome composition data element (e.g., data element 30A, as shown in FIG. 3A) by embedding the preprocessed microbiome composition data element (e.g., data element 30A, as shown in FIG. 3A) into a latent feature space (e.g., feature space 40A, as shown in FIG. 3A) using a fully connected neural network (FCNN) (e.g., network 40, as shown in FIG. 3A), said latent feature space (e.g., feature space 40A, as shown in FIG. 3A) being representative of a microbiome-metabolome interaction. Step S1015 may be carried out by FCNN 40 (as described with reference to FIG. 3A).

As shown in step S1020, the at least one processor (e.g., such as processor 2 of FIG. 2) may predict said at least one biological property (e.g., property 70A and/or concentration 60B, as shown in FIG. 3A) of the host organism, based on the obtained intermediate latent representation (e.g., representation 40B, as shown in FIG. 3A). Step S1020 may be carried out by metabolite concentration calculation module 60 and ML-based condition prediction model 70 (as described with reference to FIG. 3A).

Various aspects of system and method as suggested herein are further explained in greater detail in relation to the conducted research, tests and experiments, referring to specific examples of system configuration, and specific method implementations, which shall not be considered exclusive and limiting the scope of the present invention, as defined by the appended set of claims.

In the conducted research, the analysis included two main stages: first, the prediction of the metabolite concentration from the microbe frequencies and the resulting latent representation; second, the prediction of the host condition is based on this representation. Multiple models were proposed for both stages.

Linear Models. Different approaches have been proposed in recent years to link the microbiome composition with metabolomic data. One strategy relies on the creation of a connection network linking a given gene/amplicon sequence variant (ASV)/taxon to pathways and compounds in a database. These linkages are used to infer molecular compound identities from the genetic composition of the microbial community. Most methods are descriptive. However, there are some quantitative methods. Such methods include predicted relative metabolomic turnover (PRMT) to predict metabolites from a coastal marine metagenomics dataset, showing a clear correlation between the predicted metabolites and environmental factors. MIMOSA was later developed to predict metabolic potential in a given microbial community and to identify the microbial taxa most associated with the synthesis/consumption of key metabolites. Both methods rely on a reaction network and are limited to the KEGG database. A similar method to predict metabolites directly is Mangosteen, a metabolome prediction pipeline dependent upon relationships between KEGG/BioCyc reactions and the molecular compounds directly associated with those reactions. All the above methods are reference-based, and as such rely heavily on the completeness and accuracy of the database query. MelonnPan uses ML to predict metabolomic potential scores, which represent the relative capacity of the community in a given sample to generate or deplete each metabolite. MelonnPan has good accuracy on a specific IBD dataset.

Different ML-based models. Similar to MelonnPan, MiMeNet is an MLPNN (multiple-layer perceptron neural network) model that is composed of multiple fully connected hidden layers. They further define well-predicted metabolites. Various methods adopt the encoder-decoder paradigm, for example, Sparse-NED-a sparse one-layer neural encoder-decoder network predicts metabolite abundances from microbe abundances and Khajch et al. multi-task autoencoder to extract the latent profiles from the combined microbiome and metabolome data for IBD prediction. A more intricate example is mNODE (metabolomic profile predictor using neural ordinary differential equations) which is a deep learning method that combines explicit layers with implicit layers where the states of hidden layers are described by ODEs.

Most current methods are database-specific and cannot be trained on one cohort and tested on another cohort. In other words, they are not transferable. There was a single attempt to perform cross-predictability between datasets by a random forest regression model. Unfortunately, their success was limited to specific metabolites in specific pairs of datasets.

Prediction of host condition based on combination of metabolome and microbiome. A distinctive perspective is to use the combined microbiome-metabolome to predict the host condition. Such an approach is adopted by the Multiview model, wherein the microbiome and metabolites are treated as distinct perspectives of the host condition (assumed to be the environment affecting the microbiome and metabolome). Multiview uses “Cooperative Learning,” which combines the standard squared-error loss with an “agreement” penalty to encourage the predictions from different data views (microbiome and metabolites) to agree. Another novel approach is the IntegratedLearner, which applies Bayesian ensemble methods to consolidate predictions by harnessing information across multiple longitudinal and cross-sectional omics data layers.

The conducted research demonstrated the results discussed below.

Relation between microbiome and metabolites is not linear and is dominated by a few taxa. First, the linear model was tested. In this model, the relation between metabolites and microbiomes is often described through a consumption/production network. Such networks are based on three assumptions: A) Each microbe may consume more than one metabolite or produce different metabolites, and therefore such a network is required. B) Production and consumption rates are not frequency dependent. As such, one could assume the concentration of the metabolites would depend on a linear combination of their production and consumption by a variety of different microbes. This assumption may fail following non-linear experimental response curves for both microbes and metabolites or non-linear consumption/production. C) The relation production and consumption rate are not affected by external factors or other bacteria.

To test the first assumption, an NMF (Non-negative matrix factorization) decomposition (see detailed description below) of the metabolite non-negative concentrations (relative normalized) over the microbial relative frequencies of 10 paired microbiome-metabolome datasets (5 16S rRNA gene sequencing-based and 5 WGS datasets (see detailed description below)) was performed. Surprisingly, in most cases (94.9%), a single microbe was associated with more than 80% of the production of a single metabolite, as measured by the NMF coefficient values.

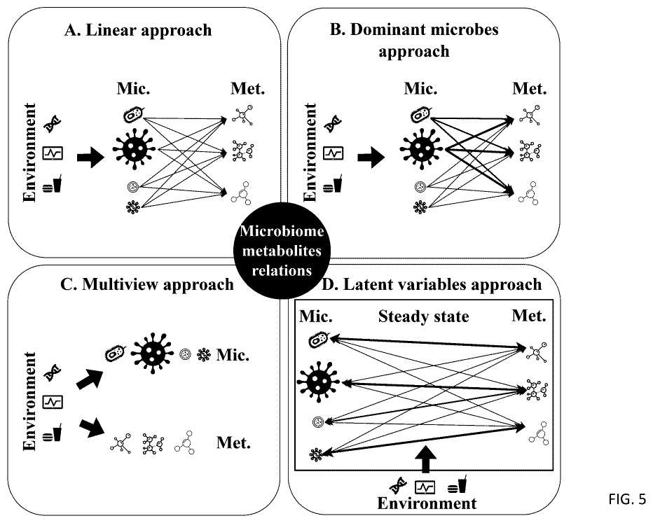

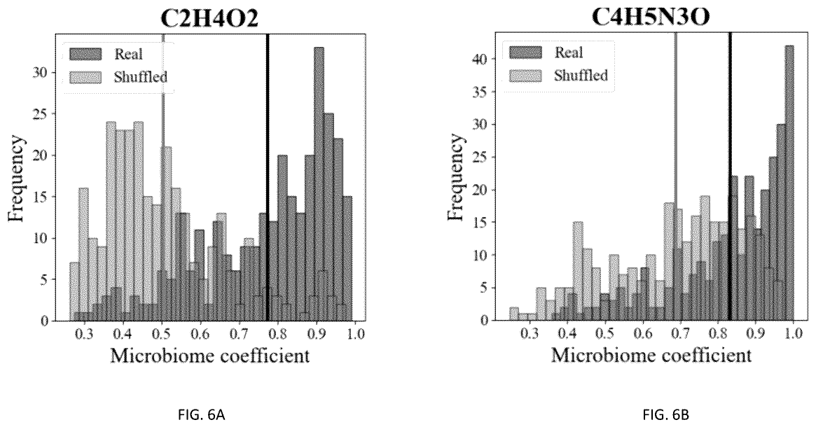

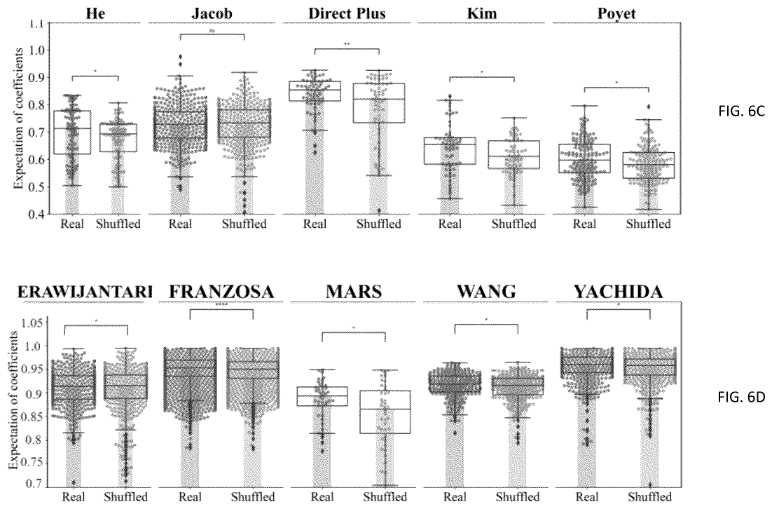

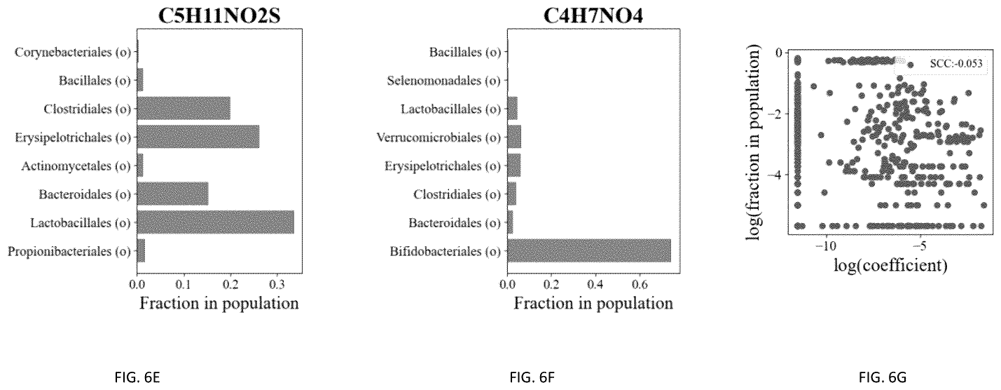

To test that such a skewed effect is not a direct result of the microbiome and metabolome distributions, the real model was compared to the relative contribution of the coefficients of a random parallel model whose microbes are shuffled (FIGS. 6A-6B, the expectations are in black (real) and grey (shuffled) for the averages of all coefficients' relative contributions of all metabolites in the “He” dataset) as well as significantly higher coefficient relative contributions expectations for each metabolite (p-value <0.05) for all datasets (FIGS. 6C, 6D). As such, the concept of a metabolite-microbe interaction network (linear approach) may fail, and instead, a direct relation between a dominant microbe and a metabolite should be considered (FIGS. 6A-6D). Thus, the dominant microbe described above is more consistent with the observed interaction than the linear approach.

If follow the dominant microbes approach, one could presume that the dominant microbes would be the very frequent ones. However, this is not the case. In the 10 most dominant taxa per metabolite (with the highest coefficients), when computing their frequency over the population, rare taxa are often dominant (FIGS. 6E, 6F), the SCC between the microbe's NMF coefficient and their fraction in the population is typically null −0.05 (FIG. 6G). To summarize, neither the linear nor the frequent dominant microbes approach seems to be consistent with the observations. Consequently, we propose to use a non-linear model relating to the log microbe frequencies and the log metabolite concentrations (FIGS. 6E, 6F, 6G), instead of a linear model or single dominant microbes models.

It was determined that latent representation of a microbiome (LOCATE) can be used to predict metabolites in each dataset separately better than all existing methods.

Given the frequent contribution of a single yet varying microbe to each metabolite's concentration, it was tested whether a relation between the log of the microbe composition and the log of the metabolite concentration would produce a better prediction (further referred to as the “Log network” model). The Log network model assumes that a matrix A connects the logged microbiome frequencies Mi to the logged metabolites concentrations, Me (similar to the approach shown FIG. 1A). To find this matrix and avoid an over-fit, Singular Value Decomposition (SVD) and a low-rank approximation (similar to the approach shown in FIG. 1A) were applied on its result. The resulting low-rank approximated matrix A* (FIG. 1A) is multiplied by the log microbe frequencies to produce the concentration of the metabolites (FIG. 1A).

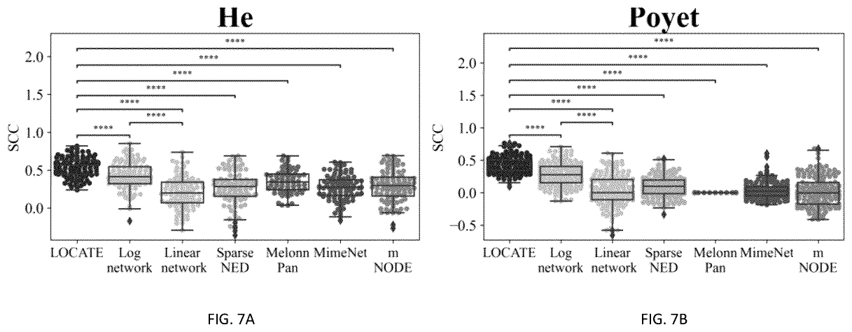

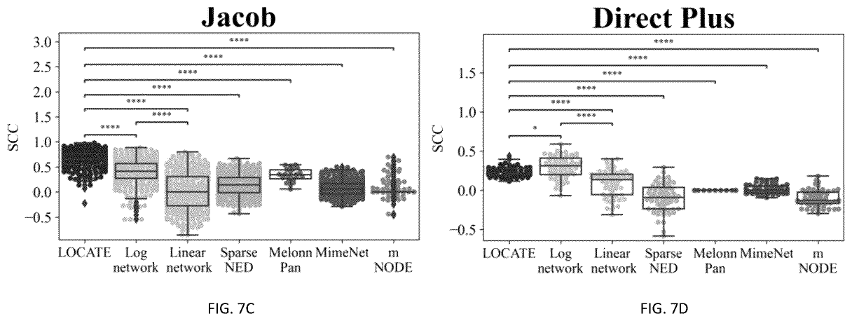

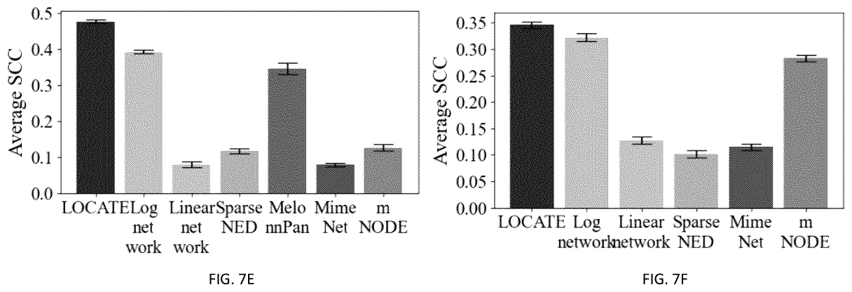

A representation of the log of each metabolite as a linear combination of the log of the microbial taxa frequency would imply a purely multiplicative relation between microbes and metabolites. While this produces a significantly (p-value <0.05) more accurate prediction than the linear relation (for 16S FIG. 7A-7E light blue vs. light grey and for WGS FIG. 7F), it is also a non-realistic assumption. In order to produce a more realistic model, it was tested to translate the log microbiome into an intermediate representation through a neural network (latent variables approach) (FIG. 1A), and then relate this representation to the log metabolome assuming a linear relation. This model is denoted herein as LOCATE-Latent variables Of microbiome And meTabolites rElations (also referred herein as system 100, with reference to FIGS. 1A-1B and 3A-3C). Formally, a latent representation of the microbiome (Z), is computed by a fully connected network (FCNN) (FIG. 1A). Then, a similar solution to the Log network model is applied to the intermediate representation of the microbiome (Z) to translate Z into the logged metabolites, (Me) (FIG. 1B). Z may then be further used to predict the host condition. Note that this entire model is trained at once. Thus Z is inherently trained to represent the relation between the microbiome and the metabolome.

To evaluate LOCATE, the SCC was measured between the real and predicted metabolites over 5 different 16S rRNA gene sequence-based datasets with 11 phenotypes and 5 different WGS datasets with 5 phenotypes. The results were compared to existing state-of-the-art models, such as MelonnPan, MimeNet, SparseNED and mNODE as well as to a Linear network and a Log network model. LOCATE significantly outperforms the state-of-the-art-models (p-value <0.001) on each dataset separately (for 16S FIGS. 7A-7D) and on the average of all the datasets and all the metabolites (for 16S FIG. 7E and for WGS 7F). LOCATE also significantly outperforms the Linear and Log-log models (for 16S FIG. 7A-7E blue and grey colors, for WGS FIG. 7F).

To summarize, modeling the metabolites and microbiome relations via an intermediate latent representation is better than all the existing methods of interaction networks or direct predictions and combined learning.

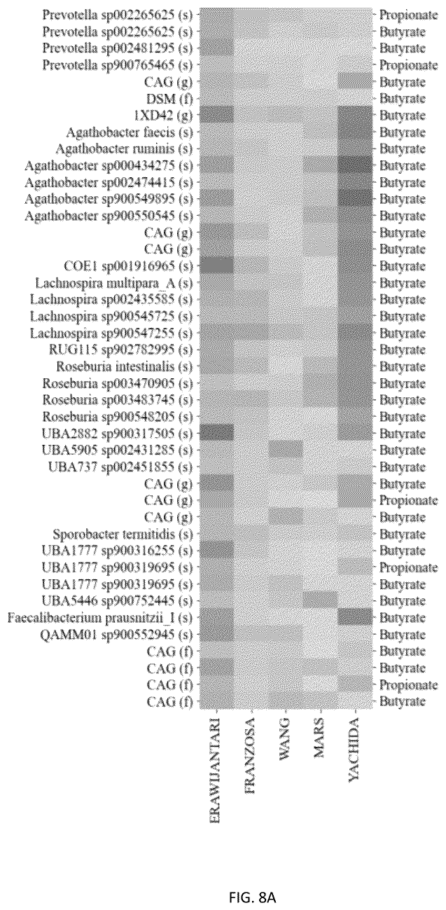

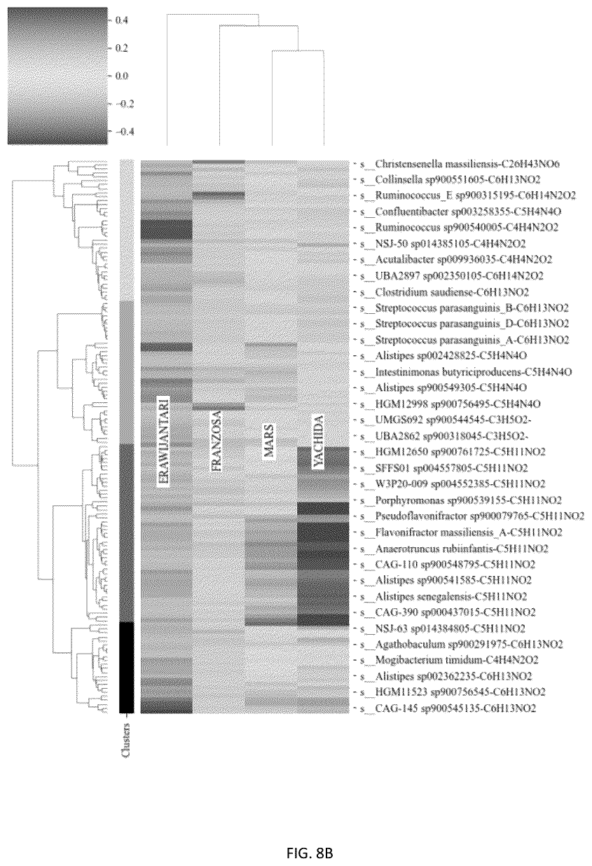

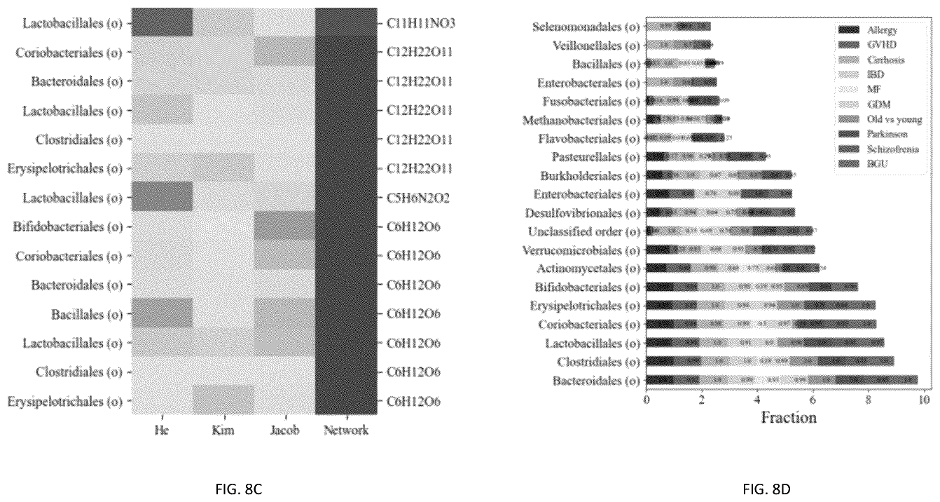

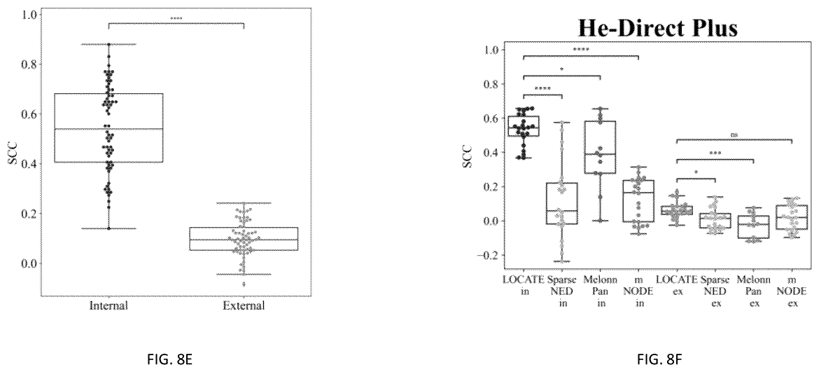

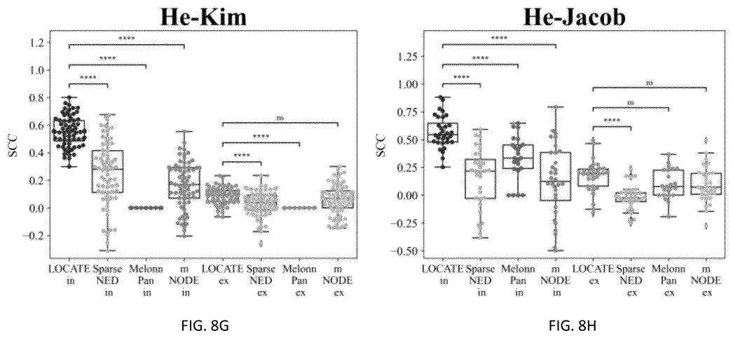

It was further determined that microbiome-metabolite relations are dataset specific. Given the high accuracy of LOCATE, it was used to test whether the relation between microbiome and metabolome is conserved between conditions and datasets, or whether it is affected by the experimental procedure and host conditions. To test dataset dependence, the association between metabolome and microbiome on the samples directly on the measured concentration was first checked. Given the importance of SCFA, it was given the priority. The SCCs between the existing SCFA in the cohort and each microbe were calculated, and significant SCCs (p-value <0.05) were considered. There are 141 different microbe-metabolites common pairs over the 5 WGS datasets. The microbial SCFA relations are indeed consistent over different datasets, with minor exceptions in several pairs especially in the WANG and MARS datasets (FIG. 8A). However, the consistency in microbes and metabolites relations is not universally conserved for all metabolites. Repeating the same computations for all metabolites over 4 WGS datasets of gastric problems ERAWIJANTARI, FRANZOSA, MARS, and YACHIDA, 4 types of pairs emerge (FIG. 8B). Most pairs are inconsistent among datasets, especially the positive correlations (the first bright grey cluster). Some are totally inconsistent (the second darker grey cluster). There are consistently negatively correlated pairs (the third darker grey cluster), and inconsistent pairs that tend to be negatively correlated (the last darkest cluster). Given the discrepancy in most pairs, it was assumed that the microbes-metabolites relations are associated with external features. An even more extreme inconsistency can be seen when comparing the data sets of the 16S (FIG. 8C). Note that this analysis was applied at the order level of the microbiome to ensure a large enough intersect between the microbes present in different datasets, which is extremely low. To further test the consistency between datasets, each metabolite-microbe pair appearing in at least two datasets was analyzed and the average SCC between each microbe-metabolite pair among all datasets containing the pair was computed. The distribution of results is very narrow, around zero (−0.003+/−0.08), suggesting that there is practically no pair with consistent positive or negative correlation. Furthermore, when comparing the raw correlations with the relations that are reported in the literature, there are many contradictions. For an example of the inconsistent correlations across datasets and differing literature, see FIG. 8C. The same phenomenon appears when comparing the weights of the Log network coefficient matrix of different datasets. Note that the correlations are at the univariate level (a single microbe vs. a single metabolite), while the coefficients are the results of a multivariate analysis.

To further test for dataset dependence, we applied cross-dataset learning, where all models are trained on one cohort and are tested on another cohort, or a cross-condition prediction, where the models are trained on one condition in the dataset and tested on another in the same dataset. To ensure that the results are not induced by the technical details of a specific model, we repeated the analysis for multiple models. When applying the cross-datasets analysis, one may encounter a technical limitation. At the order level, most of the orders are unique to a specific dataset with 17% shared orders on average between 3 datasets. The intersection between datasets is even lower at finer taxonomy levels. The overlap between pairs is higher in the WGS pairs than in the 16S pairs, especially at the species level. Surprisingly the intersection between the 16S and WGS is lower than the intersection within the 16S pairs. A quite similar situation happens in the metabolites. The average fraction of shared metabolites between the 3 datasets is 0.0114. However, there is a core microbiome of about 20 orders which appears in high amounts in most of the datasets (FIG. 8D).

To apply cross-dataset learning, one could use the microbiome common orders, defined as the core microbiome (FIG. 8D), and predict only the shared metabolites. Two kinds of learning were evaluated. The first is referred to as “in”-learning, and is based solely on the core microbiome in a given dataset. The second is referred to as “ex”-learning is applied to the core microbiome between datasets (i.e. only microbes present in high frequency in both datasets). In the “ex”-learning setup, the training is on one dataset and the testing on the other. The metabolites concentration prediction's accuracy in the “in”-learning is similar to the prediction using the entire microbiome (FIGS. 7A-7F vs. FIGS. 8F-8H “in”). However, in the “ex”-learning, the metabolites concentration prediction's accuracy over all the models is much lower (FIG. 8F-H “in” vs. “ex”).

The same decrease is observed in the Log network without the intermediate representation of LOCATE. Note that even in the cross-datasets predictions, LOCATE significantly outperforms the existing state-of-the-art models in most of the pairs both in the “in”-learning and the “ex”-learning (FIG. 8F-8H). The same decrease in accuracy is observed even in a given dataset with the same sequencing, the same machines, and the same cohort's participants in different time points (T0 vs. T6) of the Direct Plus experiment (FIG. 8E). The decrease in accuracy in the “ex”-learning of the cross-datasets analysis may result from a “context” dependent relation between the microbiome and metabolites. We propose to use this dependence to predict the host condition.

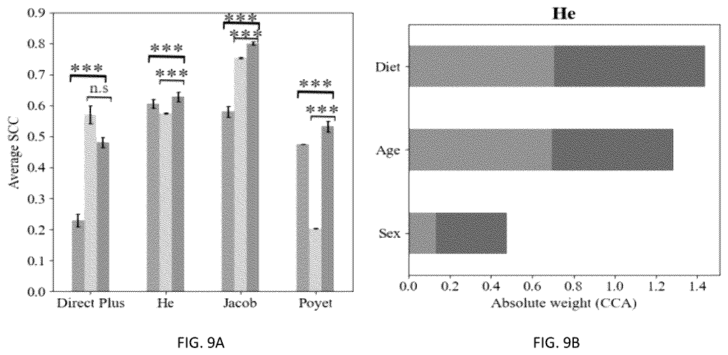

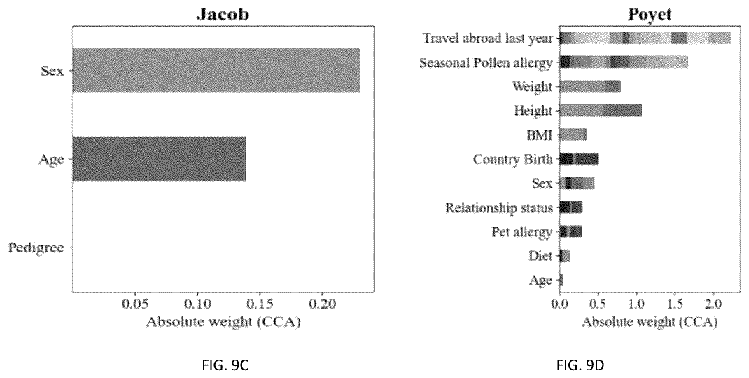

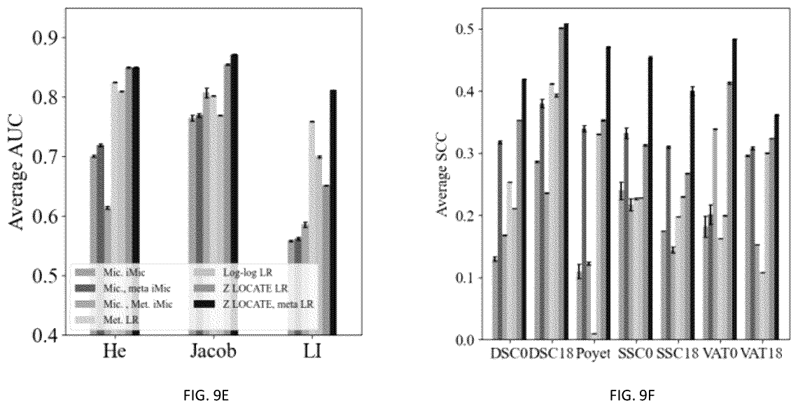

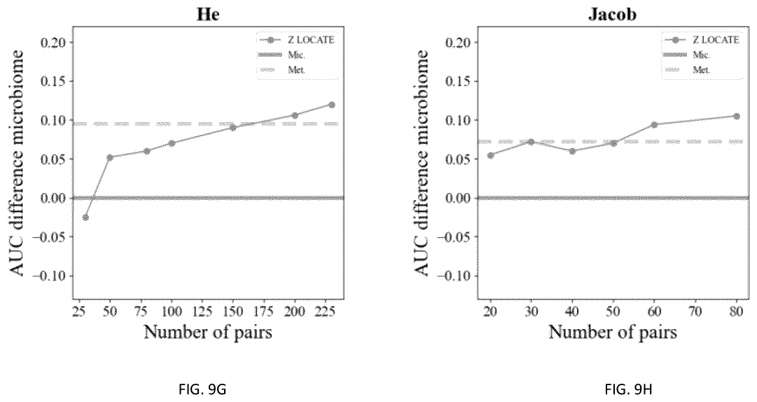

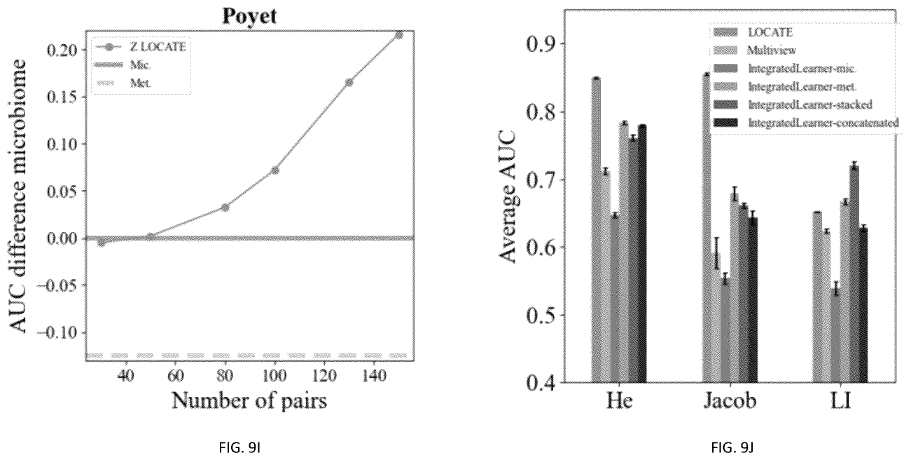

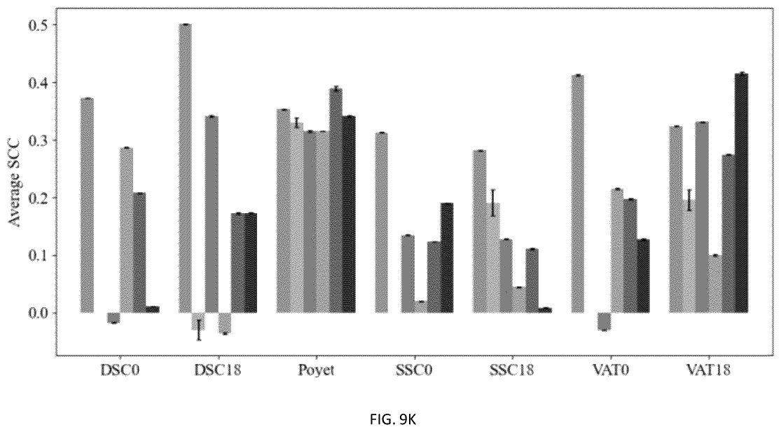

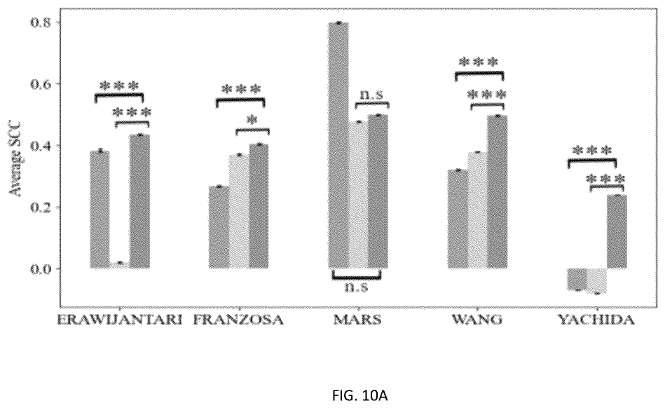

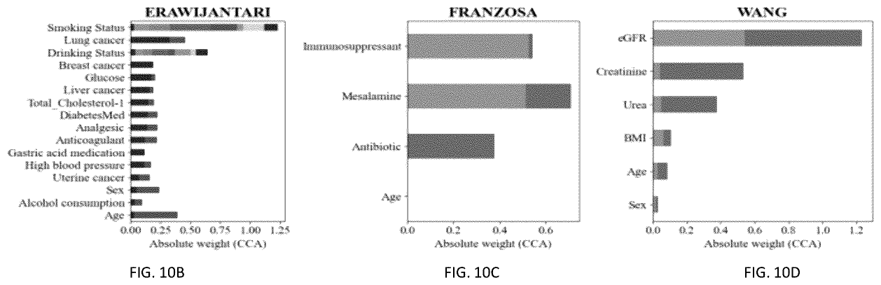

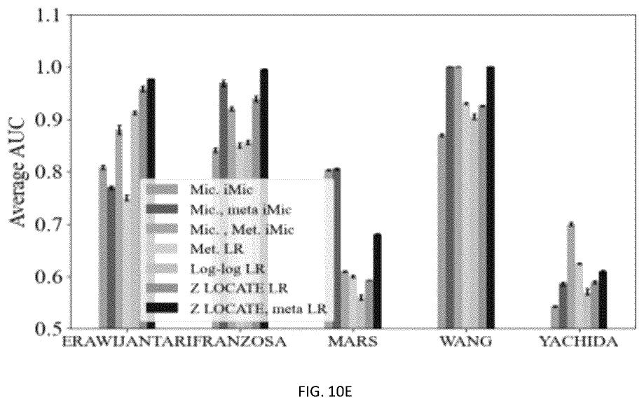

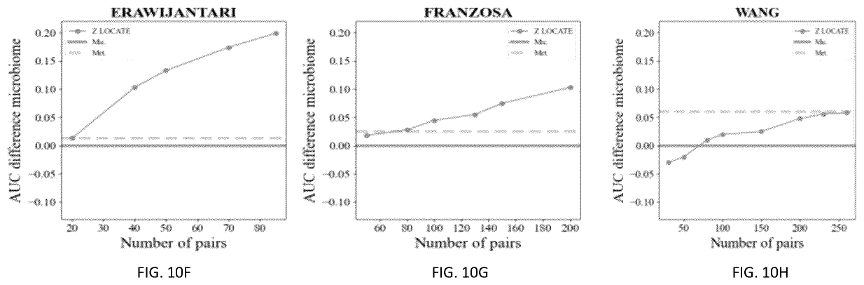

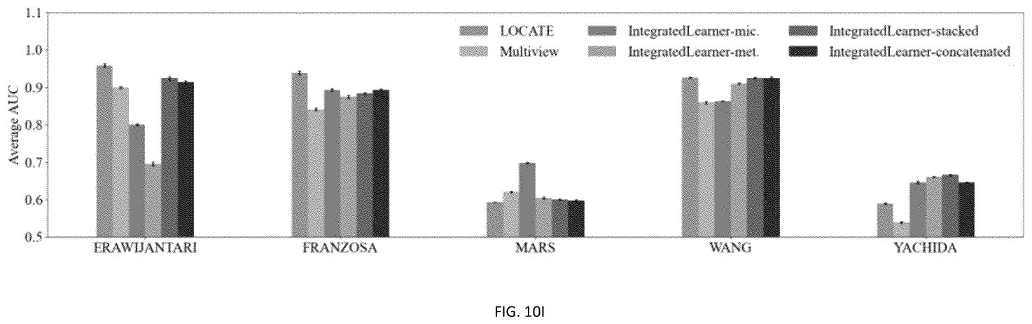

It was further determined that internal representation is associated with dataset features. To test for a relation between the latent representation (Z) and demographic and health aspects of the hosts, such as their age, sex, etc., a canonical correlation analysis (CCA) was applied to relate either the microbiome or the metabolites or the latent representation to the available host characteristics. Then the SCC was calculated between the first component of the CCA of each pair. The highest SCCs are obtained in the pairs of the representation vector and metadata with p<0.001 in most of the comparisons (for 16S FIG. 9A and for WGS FIG. 10A). Further, the weights of the analysis of the first and second components are plotted. Quite consistently the weights of the age and sex are dominant (for 16S FIGS. 9B-9D and for WGS FIGS. 10B-10D).

Then it was established that internal representation improves host condition prediction compared with microbiome or metabolome separately or their combination.

The stronger association of Z with demographics than either microbiome or metabolome by themselves may suggest that it can be a better predictor of host condition of interest in different experiments. To test for that, the host condition prediction accuracy of different outcomes from binary conditions such as healthy vs. ill (e.g., IBD in the Jacob and FRANZOSA datasets, IBS in the MARS dataset, CRC in the ERAWIJANTARI and YACHIDA dataset, fatty liver, LI, in the Direct Plus dataset, and ERSD in the WANG dataset, infants diet (breast-feeding vs. formula) in the He dataset) was compared to continuous conditions, such as age in the Poyet dataset and amounts of fats DSC, SSC, and VAT at different time points of the experiment in the Direct Plus dataset.

The prediction of cohort outcomes from LOCATE's internal representation has a higher AUC/SCC for binary/continuous predictions than the microbiome-based predictions and most of the times also of the metabolites (FIG. 9E, 9F for 16S and FIG. 10E for WGS). For the microbiome-based predictions, logistic regression was applied on both the order level and iMic, which is the state-of-the-art in microbiome predictions on the species level. The LOCATE model was applied to both the microbiome of the order level and the species level, without significant differences between them. A logistic regression model was applied to LOCATE's internal representations to predict the outcomes. The LOCATE's representations are significantly more accurate (in most of the datasets and tasks apart from the Direct Plus, LI) with p-value <0.001 better than using only the microbiome or only the metabolites as predictive features.

One may propose that Z is basically equivalent to demographic or additional data available in the different datasets, and as such is not useful beyond this demographic data. To test that this is not the case, we predicted the condition using a combination of Z and demographic data, The combination of Z and the additional data available for each data set has higher accuracy than either by themselves or from the microbiome with the additional data (for 16S FIG. 9E, 9F and for WGS FIG. 10E). To summarize, it has been shown that the latent representation Z is associated with the demographic properties of the host but adds more information to it about the condition than either the microbiome or metabolome.

Moreover, predicting the host condition based on the intermediate representation (that consists of the microbiome-metabolome interactions) is better (apart from LI in the 16S) than predicting the condition from the predicted metabolites of the Log network model (FIGS. 9E, 9F) as well as from the combined microbiome and metabolites model (apart from YACHIDA in the WGS, FIG. 10E).

Often, given the cost difference between metabolome profiling and microbiome sequencing, one aims experimentally to measure the metabolite concentrations on a sub-group of the samples. Given the fact that once the model is trained, Z is only computed using the microbiome, one may propose to measure metabolites and train the model on a partial set of samples, and then compute Z for all the microbiome samples. To test for such a hybrid sampling method, we computed the minimal number of samples required for training LOCATE's representation, such that the prediction accuracy would be higher than the one of the microbiome on the test set. For most datasets, using 50 metabolite samples and above for the training is enough to improve the overall accuracy (for 16S FIGS. 9G-9I and for WGS FIGS. 10F-10H). Note that the improvement in condition prediction presumes a relation between the metabolites and the condition. Therefore, it is recommended to first check for this relation through the metabolite reconstruction accuracy, and only then to apply LOCATE to predict a condition.

To get a more holistic comparison, LOCATE's results were also compared to other multi-omics approaches, such as Multiview and IntegretaedLearner both on 16S cohorts and WGS cohorts. LOCATE significantly outperforms the state-of-the-art multiview approaches (for 16S FIGS. 9J-K and for WGS FIG. 10I).

A discussion of the suggested techniques and of the test results is provided further below.

Two different tasks are often performed when combining microbiome and metabolome studies. The first is to predict the metabolome from the microbiome (the opposite is rarely done, apart from a single work of predicting the gut microbiome alpha diversity from the metabolites compositions), and the second is to combine both metabolome and microbiome to predict a phenotype of the host or any other property of each host. The first was typically done by a linear or non-linear translation of the microbiome into some or all of the metabolites (sometimes only a subset of the metabolites in the sample are predicted), assuming that the microbiome determines the metabolite concentration. The second task is typically performed by combining the two types of data and performing a prediction of the target condition.

It is proposed herein that the first task should be treated through the creation of a latent representation (Z) of the microbiome and metabolome, using LOCATE. This representation is host and condition-specific. This representation is associated with the sample context which can be the age, gender, dietary habits, or health condition of the host. We then show that Z is strongly associated with the host demographic, diet, and other features. Finally, we show that it better predicts the host properties than either the microbiome or metabolome. As such it serves to combine the two tasks above. The main difference between this approach and most existing combinations of microbiome and metabolome to predict condition is that instead of combining the two, it is proposed to find intermediate variables between the two and use those to predict the condition. This representation is denoted Z (also referred herein as intermediate latent representation 40B or 40C, as shown in FIGS. 3A, 3C) all along and the algorithm producing it LOCATE (also referred herein as system 100 with reference to FIGS. 1A-1B, 3A-3C and method of FIG. 4).

By combining the solution on the above two tasks, LOCATE is less sensitive to the limitations of condition prediction by either microbiome or metabolome. It is more directly associated with host properties as measured by a CCA to measure host properties than either microbiome or metabolome. A crucial aspect of LOCATE is that it provides a low dimensional representation of the host (10 dimensions in the current analysis). Such low dimension makes the representation amenable to easy manipulation with no need for detailed knowledge of either microbes or metabolomes.

At the practical level, we show that it is enough even for experiments with a large number of samples to measure less than 100 metabolome samples. Those can then be integrated with the microbiome using LOCATE, and the internal representation of all other samples can be computed from the microbiome via LOCATE. As such, it can serve as a viable solution for large cohorts at a reasonable cost. This solution is applicable to both 16S and WGS.

The development of a cross-platform latent variable host representation that could merge 16S and WGS may be improved by providing domain-invariant machine learning algorithms. A classical solution would be the combination of LOCATE with an adversary classifier. Such a system could in theory distill the representation relevant to the host biology from the ones related to the experimental procedure. Such architecture has also been tested.

Beyond its application as a prediction tool for metabolite and host conditions, LOCATE can also be used to define a distance between samples since it provides an internal low-dimension latent representation. Such a distance can be used among many others for visualization through a PCoA projection to 2 or 3 dimensions. It can be used for sample clustering and anomaly detection. The analysis of the statistical properties of this distance would require more datasets than currently used and is left to further studies.

The association of microbes with metabolites is treated oversimplistically as a direct relation of microbes consuming a metabolite increasing in its presence, and similarly the concentration of metabolites produced by a microbe increasing in its presence. It has been shown that a much more complex relation should be considered, where the metabolites and microbes produce a condition-dependent equilibrium. On one hand, one cannot simply predict the change in a microbe through a change in the metabolites. On the other hand, this equilibrium allows for a precise prediction of the condition based on the combined metabolite and microbes of a set of samples. Interestingly, even with a small number of metabolites, a latent representation of the environment can be produced from the relation between microbes and metabolites. This representation can then be used with large microbiome samples to predict with high accuracy disease states or other conditions associated with the gut-metabolome.

Specific methods and techniques used for the research are discussed further below.

Datasets. Data from multiple published studies of the human gut microbiome and metabolome has been analyzed. The research focused on studies that included at least 90 individuals, following the rules proposed by Borenstein's gut microbiome-metabolome dataset collection, for which both the microbiome and the metabolome were profiled from fecal samples. The research was based on five 16S rRNA gene sequencing paired datasets and five whole genome shotgun sequencing (WGS) paired datasets.

The following 16S rRNA gene sequencing-based paired datasets.

DIRECT PLUS. Longitudinal samples of fecal microbiome and metabolites (over 18 months) of 294 participants with abdominal obesity/dyslipidemia into healthy dietary guidelines (HDG), MED, and green-MED weight-loss diet groups, all accompanied by physical activity. The outcomes we studied here were deep subcutaneous (DSC), superficial subcutaneous (SSC), Visceral adipose tissue (VAT), and fatty liver. During this analysis, we used only the microbiome and metabolites from the first time point (TO) and the last time point (T18) separately for each participant.

He. Microbiome and metabolites from infants over several time points during the first year of life, either breastfed, formula-fed, or experimental formula fed.

Jacob. IBD patients, twenty-one Crohn's disease (CD) and ulcerative colitis (UC) probands younger than the age of 18 were recruited from the Pediatric IBD Center at the Cedars-Sinai Medical Center and their first-degree relatives of patients with IBD. Both their microbiome and their metabolites of them were analyzed.

Kim. Fecal microbiome and metabolites of patients with advanced colorectal adenomas, colorectal cancer, and controls.

Poyet. Longitudinal samples from healthy donors to the Broad Institute-OpenBiome Microbiome Library (BIO-ML).

The following WGS paired datasets were used.

ERAWIJANTARI GASTRIC CANCER 2020. Fecal and metabolites of patients who underwent colonoscopy, half of whom with a history of gastrectomy for gastric cancer and no signs of gastric cancer recurrence. This dataset is referred to as ERAWIJANTARI. FRANZOSA IBD 2019. Fecal microbiome and metabolites of IBD patients and controls (PRISM cohort+A validation cohort). This dataset is referred to as FRANZOSA.

MARS IBS 2020. Longitudinal samples fecal microbiome and metabolites (over 6 months) from patients with Irritable Bowel Syndrome (IBS) and controls. This dataset is referred to as MARS.

WANG ESRD 2020. Fecal microbiome and metabolites of adults with end-stage renal disease (ESRD) and controls. This dataset is referred to as WANG.

YACHIDA CRC 2019. Fecal microbiome and metabolites of patients who underwent colonoscopy, with findings from normal to stage 4 colorectal cancer. This dataset is referred to as YACHIDA.

Microbiome preprocessing. For LOCATE, microbial data was pre-processed using the MIPMLP pipeline. We merged the ASVs either to the order (to gain maximal intersection between pairs) or to the species taxonomic level by the Sub-PCA method (detailed below), but similar results are obtained at the other taxonomy levels as well (data not shown). Then, log normalization (detailed below) on the merged ASVs was applied. Further, each taxon was normalized such that its average was 0 and its variance was 1 (z-score). Notably, variations of the LOCATE model with no z-scoring for the microbial data was also tested, yet for clarity, outcomes from the highest accuracy variant are presented herein. For the other algorithms (SparseNED, MelonnPan, mNODE, and MiMeNet), the research followed the preprocessing that was reported in the relevant papers. For the pre-analyses of the approach of FIG. 1A-1B, and 6A-6G), the ASVs were merged to the order level by the mean method (detailed below), and a relative normalization (detailed below) was applied (to keep the values positive).

Sub-PCA merging in MIPMLP. A taxonomic level (e.g., species) was set. All the ASVs consistent with this taxonomy were grouped. A PCA was performed on this group. The components which explain more than half of the variance are added to the new input table. This merging was applied for LOCATE.

Mean merging in MIPMLP. A level of taxonomy (e.g., species) was set. All the ASVS consistent with this taxonomy were grouped by averaging them. This merging was applied to the NMF and to the analyses of FIGS. 6A-6G.

Relative normalization in MIPMLP. To normalize each taxon through its relative frequency

$${x}_{i , j} = \frac{{x}_{i , j}}{{\sum }_{k = 1}^{n} {x}_{k , j}} ,$$

the relative abundance of each taxon j in sample i was normalized by its relative abundance across all n samples. This was applied only to the NMF model and to the analyses of FIGS. 6A-6G.

Log normalization in MIPMLP. The process included logarithmic (10 base) scaling of the features element-wise, according to the following formula:

x_{i,j}→log(x_{i,j}+ε),
where ε is a minimal value (=0.1) to prevent log of zero values. This was applied for LOCATE.

Metabolites preprocessing. For LOCATE, all metabolic samples were first normalized to relative frequencies, such that the metabolites of each sample would sum to 1. Then those were log-normalized and further z-scored, such that the average value of each metabolite would be 0 and its variance would be 1. Again, for the other algorithms, the research followed the preprocessing that was reported in the relevant papers. For the analyses of FIG. 6A-6G, only relative normalization was applied.

Matrix factorization methods. A Non-Negative Matrix Factorization (NMF) was used, that finds two non-negative matrices (W, H) whose product approximates the non-negative matrix Me. This factorization can be used for example for dimensional reduction, source separation, or topic extraction. In the present case, it was assumed that Meₜᵣₐᵢₙ is the non-negative metabolites matrix of the training, and it was expressed as a product of the training microbiome matrix (Miₜᵣₐᵢₙ) and another relations matrix (A). Then the training relations matrix was used to predict the metabolite values from the microbial abundances. The NMF decomposition of sklearn version 0.24 with its default parameters apart from the L1 regularization that was fine-tuned and set to 10 was used herein. The initialization matrix was initialized randomly with numbers between 0 to 1. It was checked that different initializations do not affect the convergence of the algorithm.

Metabolites prediction by Latent variables Of microbiome And meTabolites rElations (LOCATE). To predict the log normalized metabolite concentrations (Me, FIGS. 1A-1B) from the log normalized microbiome (Mi, FIGS. 1A-1B), we first built an intermediate latent representation between the microbiome and the metabolites by using a fully connected neural network (Z, FIG. 1A). Representation network. A 3-layer fully connected neural network FCNN was applied to the log-normalized microbiome data (different dimension reduction methods, such as ID-CNN and deep networks were also tested). An activation function (either of RelU, elU, or Tanh) was applied between the layers, and dropout and L2 regularization were also applied. The representation dimension was set to 10. All the network hyperparameters, except for the representation dimension, were chosen via the Neural Network Intelligence (NNI) platform on each dataset separately on an internal validation set. The loss was a standard MSE loss

$$\frac{1}{N} {\sum }_{i = 1}^{N} \sum {( {Me}_{i} - {ι}_{} )}^{2} ,$$

where N is the number of the microbiome and metabolites paired samples and [symbol] is the predicted metabolites, and an Adam optimizer was used (FIG. 1A).

The output of the neural network was the input of a linear predictor of the log metabolite concentration. We assumed a microbiome-metabolites relationship matrix A (FIG. 1A) such that

$$A \cdot Z = Me \to A = {Z}^{- 1} \cdot {Me}_{train} ,$$

where Z⁻¹ is the pseudo inverse of Z ((Z^{t}·Z)⁻¹·Z^{t}), since Z does not have to be a squared matrix. The pseudo-inverse was computed using the torch.linalg.Istsq function on Z and Meₜᵣₐᵢₙ (FIG. 1A). To prevent overfitting, we did not use A directly but applied a low-rank approximation on A using torch.svd_lowrank with its default parameters (FIG. 1A). In the inference step, the low-rank approximated matrix of A* (an approximating matrix with reduced rank) from the training was used (FIG. 1B). It is important to note that the neural network's end-to-end training produces a representation (Z) that encodes the combined information of both microbiome and metabolites (via the backpropagation), even though its direct connection to metabolites might not be as explicit as that of A. LOCATE was also tested without the low-rank approximation.

Host condition prediction. To test which variables best explain the condition of the cohorts, we predicted the condition once from the original microbiome (Mi) at two different taxonomy levels, the order level, and the species level, once from the original metabolites (Me) and once from LOCATE's latent microbiome-metabolites representation (Z). For binary conditions, a logistic regression model was applied with its default parameters, including an L2 regularization of 1, of the sklearn library. For continuous conditions, a Ridge regression model was applied with its default parameters of the sklearn library. Note that no hyperparameter tuning was applied. The data was split into a training set (80% of the data) and a test set (20% of the data), and we reported the results on the test set as an average of 10 different splits as described in the Statistics and evaluation section. For the microbiome-based learning at the species taxonomic level, the microbiome was translated into a 2D image, such that each row of the image represents another taxonomy level according to the cladogram structure. Then a novel CNN-based prediction—iMic was applied for both the regression and classification models.

Statistics and evaluation. Spearman Correlation Coefficient (SCC) and Area under the receiver operating characteristic (ROC) Curve (AUC).

To evaluate the prediction quality of the metabolite predictions, we calculated the Spearman Correlation Coefficient (SCC) between the real metabolites and the predicted metabolites over two different frameworks: within a given dataset-by removing 20% of the data for testing, and cross-datasets approach, training the model on one dataset and testing it on another.

SCC was further used to evaluate the condition prediction of the continuous conditions by measuring the SCC between the predicted phenotype and the real phenotype on the test set. An average of 10 cross-validations on the test set was reported.

To evaluate the condition predictions of the binary phenotypes, the AUC of each model (microbiome-based, metabolites-based, and representation-based) was calculated on a test set. An average of 10 cross-validations was reported on the test.

Representation matrix and metadata relationships. To test the relations between the demographic features (e.g., age, gender, etc.) of each cohort and its representation (Z), we first applied Canonical-Correlation Analysis (CCA) between the original microbiome (Mi) input and the metadata, the original metabolites (Me) data and the metadata and the representation (Z). Then, we trained 10 CCA models on each of the training sets (80% of the data each) and predicted the CCA for each of the 10 test sets (20% of the data each) separately. Then the average SCCs (over the 10 models' partitions) between the real CCAs and the predicted ones were computed and reported with their standard errors over the 10 runs. We further predicted the metadata once from the microbiome (Mi), once from the metabolites (Me), and once from the representation (Z) using a Ridge model. The average SCC between the predicted metadata and real metadata was computed on the test set. To detect the metadata features that are related to the microbiome-metabolites representation (Z), the absolute CCA's weights of the first two components were computed.

### Experimental Design.

Generate a uniform platform for metabolites. Each dataset had a different notation for the metabolites. Consequently, we translated the identity of each metabolite to its chemical formula by using the API of the following websites Metabolomicsworkbench, and KEGG COMPOUND Database as well as by the PubChemPy python package.

### Training and Test Sets Split.

Representation learning. Within dataset. The data was divided with an external test of 20% of the whole data. The remaining 80% of the data was used as the training set. The split was repeated 10 times, such that the reported results were an average of the 10 runs.

Cross-datasets prediction. First, the datasets were merged by removing microbes and metabolites that did not appear in the intersection of the datasets. Then each dataset was normalized separately. Next, two different types of learning were applied: (1) “in”-learning—given one single dataset with only its core microbiome and shared metabolites, applying learning within the dataset by dividing it into a training set (80% of the data), and a testing set (20% of the data). (2) “ex”-learning—where one dataset was used for training, while the other dataset was used for testing.

Condition predictions. The data (microbiome and condition, or metabolites and condition, or LOCATE's representation, Z, and condition) was divided into 2 groups; 80% of the data was used for training and the remaining 20% was used for testing. We repeated the split 10 times, such that the reported results were an average of the 10 runs.

Creating the representation on a varying number of training samples. The research aimed to determine the optimal sample size for pairs of microbiome and metabolite data that would yield a robust microbiome-metabolite representation through LOCATE, thus enhancing condition prediction accuracy. To this end, the investigation involved varying numbers of microbiome-metabolome pairs (for example ranging from 25 to 225 in the He dataset), mirroring the common scenario where there are fewer metabolite data samples than microbiome samples. This diverse sample range was chosen to reflect the typical scope of experiments conducted. The suggested approach involved training the representation network of LOCATE, using the specified number of paired samples. Subsequently, the representations were used or all samples within the cohort, even those lacking metabolite data, leveraging the trained model.

As can be seen from the provided description, the claimed invention represents a system and method for predicting one or more properties of a host organism using microbiome composition data that provide an improvement of the technological field of computational biology by increasing the accuracy and reliability of such predictions, including the prediction of metabolome concentrations based on the host's microbiome data. The present invention provides a solution that accurately leverages the complex, non-linear, and bi-directional interactions between the microbiome and metabolome of the host. This solution considers the dynamic relationships between different microbial species and their collective impact on the host's metabolic profile.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method for predicting at least one property of a host organism, by at least one processor, comprising:
analyzing a biological sample of the host organism to obtain a microbiome composition data element comprising information about frequency values of various microbial taxa characterizing the biological sample;
preprocessing the microbiome composition data element by grouping and normalizing said frequency values, based on a microbial taxonomic classification;
obtaining an intermediate latent representation of the preprocessed microbiome composition data element by embedding the preprocessed microbiome composition data element into a latent feature space using a fully connected neural network (FCNN), said latent feature space being representative of a microbiome-metabolome interaction;
predicting said at least one property of the host organism, based on the obtained intermediate latent representation.

2. The method of claim 1, wherein said preprocessing comprises:
selecting a taxonomy level of a microbial taxonomic classification;
merging frequency values of microbial taxa belonging to a same taxonomic group of the selected taxonomy level;
based on the merged frequency values, applying Principal Component Analysis (PCA) on each taxonomic group of the selected taxonomy level, to determine those of the microbial taxa that explain at least half of a variance in the merged frequency values; and
forming the preprocessed microbiome composition data element, based on the determined microbial taxa.

3. The method of claim 2, wherein said forming of the preprocessed microbiome composition data element comprises applying a logarithmic normalization to frequency values of the determined microbial taxa, so as to reduce the impact of highest frequency values and prevent zero frequency values.

4. The method of claim 1, wherein said latent feature space comprises latent microbiome features mapped to the concentration of one or more metabolites by a predefined function.

5. The method of claim 4, wherein said predicting the at least one property comprises determining a metabolite concentration of the host organism by applying an approximated microbiome-metabolite relationship matrix to the intermediate latent representation, said approximated microbiome-metabolite relationship matrix comprising parameters of said predefined function.

6. The method of claim 5, wherein said predefined function is a linear function from the features of the latent feature space to logarithmically normalized values of said metabolite concentration.

7. The method of claim 6, wherein said applying the approximated microbiome-metabolite relationship matrix comprises multiplying the intermediate latent representation by the approximated microbiome-metabolite relationship matrix, to obtain said logarithmically normalized values of said metabolite concentration.

8. The method of claim 4, wherein said predicting the at least one property comprises inferring a machine-learning (ML)-based condition prediction model on the intermediate latent representation, said ML-based condition prediction model being pretrained to predict said at least one property based on the latent microbiome features.

9. The method of claim 8, wherein said property is determined by a binary value and the ML-based condition prediction model is a logistic regression model; or said property is determined by a continuous value and the ML-based condition prediction model is a Ridge regression model.

10. The method of claim 5, wherein the approximated microbiome-metabolite relationship matrix is obtained by:
based on a training set of paired training samples comprising training microbiome composition data elements and training metabolite concentration data elements, training the FCNN to determine the latent feature space, while concurrently determining an initial microbiome-metabolite relationship matrix comprising parameters of an initial function mapping (i) inverse latent representation matrices obtained by applying a pseudo-inverse algorithm on intermediate latent representations of training microbiome composition data elements, said intermediate latent representations of training microbiome composition data elements being calculated by embedding said training microbiome composition data elements into the determined latent feature space; to (ii) training metabolite concentration data elements of respective paired training samples; and
applying a low-rank approximation of the initial microbiome-metabolite relationship matrix using Singular Value Decomposition (SVD) algorithm, to obtain the approximated microbiome-metabolite relationship matrix.

11. The method of claim 1, further comprising, based on a training set of paired training samples comprising training microbiome composition data elements and training metabolite concentration data elements, training the FCNN to determine the latent feature space assuming a correlation with an initial function mapping (i) inverse latent representation matrices obtained by applying a pseudo-inverse algorithm on intermediate latent representations of training microbiome composition data elements, said intermediate latent representations of training microbiome composition data elements being calculated by embedding said training microbiome composition data elements into the determined latent feature space; to (ii) training metabolite concentration data elements of respective paired training samples.

12. The method of claim 11, further comprising concurrently determining an initial microbiome-metabolite relationship matrix comprising parameters of said initial function.

13. The method of claim 1, wherein said microbiome composition data element is represented by at least one of (i) 16S rRNA gene sequencing; and (ii) Whole Genome Shotgun Sequencing (WGS).

14. A system for predicting at least one property of a host organism, the system comprising: at least one non-transitory memory device, wherein modules of instruction code are stored, and at least one processor associated with said at least one memory device, and configured to execute the modules of instruction code, whereupon execution of said modules of instruction code, the at least one processor is configured to:
analyze a biological sample of the host organism to obtain a microbiome composition data element comprising information about frequency values of various microbial taxa characterizing the biological sample;
preprocess the microbiome composition data element by grouping and normalizing said frequency values, based on a microbial taxonomic classification;
obtain an intermediate latent representation of the preprocessed microbiome composition data element by embedding the preprocessed microbiome composition data element into a latent feature space using a fully connected neural network (FCNN), said latent feature space being representative of a microbiome-metabolome interaction; and
predict said at least one property of the host organism, based on the obtained intermediate latent representation.

15. The system of claim 14, wherein said at least one processor is configured to preprocess the microbiome composition data element further by:
selecting a taxonomy level of a microbial taxonomic classification;
merging frequency values of microbial taxa belonging to a same taxonomic group of the selected taxonomy level;
based on the merged frequency values, applying Principal Component Analysis (PCA) on each taxonomic group of the selected taxonomy level, to determine those of the microbial taxa that explain at least half of a variance in the merged frequency values; and
forming the preprocessed microbiome composition data element, based on the determined microbial taxa.

16. The system of claim 15, wherein said at least one processor is configured to perform said forming of the preprocessed microbiome composition data element by applying a logarithmic normalization to frequency values of the determined microbial taxa, so as to reduce the impact of highest frequency values and prevent zero frequency values.

17. The system of claim 14, wherein said latent feature space comprises latent microbiome features mapped to the concentration of one or more metabolites by a predefined function.

18. The system of claim 17, wherein said at least one property comprises a metabolite concentration; and wherein said at least one processor is configured to predict said at least one property by determining the metabolite concentration of the host organism by applying an approximated microbiome-metabolite relationship matrix to the intermediate latent representation, said approximated microbiome-metabolite relationship matrix comprising parameters of said predefined function.

19. The system of claim 18, wherein said predefined function is a linear function from the features of the latent feature space to logarithmically normalized values of said metabolite concentration.

20. The system of claim 17, wherein said at least one processor is configured to predict said at least one property by inferring a machine-learning (ML)-based condition prediction model on the intermediate latent representation, said ML-based condition prediction model being pretrained to predict said at least one property based on the latent microbiome features.
